(19) United States
(12) Patent Application Publication
(10) Pub. No.: US 2026/0139329 A1
(43) Pub. Date: May 21, 2026

(54) METHOD TO CHARACTERIZE NUCLEIC ACID

(71) Applicant: ICHORtec GmbH, Duren (DE)

(72) Inventors: Oleh Fedorych, Aachen (DE); Vitaly Matyash, Berlin (DE)

(73) Assignee: ICHORtec GmbH, Duren (DE)

(21) Appl. No.: 19/456,898

(22) Filed: Jan. 22, 2026

Related U.S. Application Data
(63) Continuation-in-part of application No. 18/889,250, filed on Sep. 18, 2024, now abandoned.

(30) Foreign Application Priority Data
Jul. 29, 2024 (EP) 24191540.4

(51) Int. Cl. C12Q 1/689; C12Q 1/6806; C12Q 1/6825; C12Q 1/6832; G01N 21/64

(52) U.S. Cl. CPC C12Q 1/689; C12Q 1/6806; C12Q 1/6825; C12Q 1/6832; G01N 21/6428; C12Q 2600/106; G01N 2021/6439

(57) ABSTRACT

The present disclosure relates to novel optical detection methods with high sensitivity for mispairing in nucleic acids. Further, the methods allow also the detection of influences of other molecules on nucleic acid duplexes. Disclosed are also uses and applications of said methods.

### CROSS-REFERENCE TO RELATED APPLICATIONS

This application is a continuation-in-part of U.S. patent application Ser. No. 18/889,250 filed on Sep. 18, 2024, the contents of which are incorporated herein by reference in their entirety. This application claims the benefit of priority to European Patent Application No. 24191540.4 filed on Jul. 29, 2024, the contents of which are incorporated herein by reference in their entirety.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 13, 2024, is named “066507.07US.xml” and is 8,000 bytes in size. The sequence listing contained in this .XML file is part of the specification and is hereby incorporated by reference herein in its entirety.

The present disclosure relates to novel optical detection methods with high sensitivity for mispairing in nucleic acids. Further, the methods allow also the detection of influences of other molecules on nucleic acid duplexes. Disclosed are also uses and applications of said methods.

### BACKGROUND OF THE DISCLOSURE

Nucleic acid hybridization is a fundamental process in molecular biology where two complementary strands of nucleic acids (DNA or RNA) bind together to form a double-stranded structure, or duplex. This process is driven by the specific pairing of nucleotide bases: adenine with thymine (or uracil in RNA) and cytosine with guanine. Hybridization plays a crucial role in various biological processes, including DNA replication, transcription, and translation, and serves as the basis for numerous laboratory techniques.

Accurate nucleic acid hybridization is essential for the integrity of genetic information. Mispairings, where non-complementary bases pair, can lead to mutations that disrupt the normal function of genes. These mutations can cause a range of genetic disorders, contribute to the development of cancers, and affect the efficacy of therapeutic interventions. Therefore, identifying mispairings and other mutations is critical for understanding genetic diseases, developing diagnostic tools, and creating targeted treatments.

Several advanced technologies have been developed to detect mispairings and mutations in nucleic acids, each with unique capabilities and applications:

Next-Generation Sequencing (NGS) provides high-throughput, precise identification of mutations across entire genomes or specific target regions. Sanger Sequencing is a more traditional method for sequencing smaller regions, offering high accuracy for detecting point mutations. Allele-specific PCR is designed to amplify and detect specific mutations by using primers that match the mutated sequence. Digital PCR quantifies the exact number of mutant DNA molecules in a sample, enhancing sensitivity.

In microarrays probes are used to detect specific sequences or mutations within a sample, allowing for parallel analysis of thousands of genetic variants. Southern blotting is a more traditional method to detect specific DNA sequences within a complex mixture by hybridizing a labeled probe to the target sequence. CRISPR-Cas9 may be utilized for precise genome editing and mutation detection, enabling the identification and correction of specific genetic errors. Other techniques exploit the natural mismatch repair system to identify and quantify DNA mismatches.

Last, but not least, does Fluorescence In Situ Hybridization (FISH) make use of fluorescent probes to detect specific DNA or RNA sequences within intact cells, allowing for the visualization of chromosomal abnormalities and gene mutations.

The ability to accurately identify nucleic acid mispairings and mutations is paramount in genetics and medical research. The development of sophisticated technologies has greatly enhanced scientists capacity to detect and analyze these genetic alterations, paving the way for advancements in diagnostics, personalized medicine, and therapeutic strategies.

However, each of the prior art methods for identifying mispairings and mutations in nucleic acids do also have their own set of disadvantages:

Next-Generation Sequencing (NGS) is expensive, complex and takes long turnaround times. Snger-sequencing is limited to small regions of the genome, time-consuming, and even more costly than NGS. Allele-Specific PCR is complex in terms or primer design and can only detect known mutations. Digital PCR is again expensive and requires specialized training and handling. Microarrays are limited in their detection profile and basically restricted to pre-defined mutations. Southern Blotting is labor-intensive and has limited ability to detect low-frequency mutations. CRISPR-Cas Systems may induce unwanted off-target effects and difficult to design.

Fluorescence In Situ Hybridization (FISH) has certain resolution limits and my not detect small mutations or single nucleotide changes. It also requires skilled personnel, can be labor-intensive and expensive.

The present disclosure presents an optical method that allows for quantitative measurements of nucleic acid hybridization and mutations at room temperature and therefore overcomes several of the disadvantages mentioned before for prior art techniques. The disclosed optical method offers quantitative measurements at room temperature and could overcome many limitations, providing a faster, simpler, and more cost-effective alternative for mutation detection and analysis. This could significantly enhance the efficiency and accessibility of genetic research and clinical diagnostics.

### BRIEF SUMMARY OF THE DISCLOSURE

In accordance with the purpose(s) of this invention, as embodied and broadly described herein, this invention, in one aspect, relates to a method for analyzing a target nucleic acid in a biological sample comprising: a) contacting the biological sample with a nucleic acid probe, wherein the nucleic acid probe is labelled with a fluorescent dye and adapted to hybridize with the target nucleic acid; b) hybridizing the nucleic acid probe and the target nucleic acid to produce a hybridized complex; c) irradiating the hybridized complex with an excitation light to excite the fluorescent dye; d) observing, with a detector, an emission of light from the fluorescent dye, wherein the emission of light has an energy of peak fluorescence (E^{S}); e) determining if the hybridized complex has one or more mispaired base pairs, comprising: i) calculating the hybridization energy difference of the nucleic acid probe hybridized to a control nucleic acid having 100% complementarity to the nucleic acid probe (ΔE^{H}) and the hybridization energy difference of the nucleic acid probe hybridized to the target nucleic acid (ΔE_{HM}), wherein ΔE^{H}=E⁰−E^{K} and ΔE^{HM}=E⁰−E^{S}; wherein E⁰ is a known energy of peak fluorescence of the nucleic acid probe; wherein E^{K} is a known energy of peak fluorescence of the nucleic acid probe hybridized to a control nucleic acid having 100% complementarity to the nucleic acid probe; and ii) comparing |ΔE^{H}| and |ΔE^{HM}|, wherein if |ΔE^{H}|>|ΔE^{HM}|, the hybridized complex comprises at least one mispaired base pair.

In some embodiments, the method further comprises calculating a change in the duplex melting temperature (ΔΔE) wherein ΔΔE=|ΔE^{H}|−|ΔE^{HM}|. In some embodiments, the method further comprises repeating steps a)-e) in the presence of a chaotropic agent; thereby determining chaotropic ΔE′^{H} and chaotropic ΔE′^{HM}, wherein ΔE′^{H}=E⁰−E^{K} and ΔE′^{HM}=E⁰−E^{S}. In some embodiments, the method further comprises calculating chaotropic ΔΔE′ by ΔΔE′=|ΔE′|−|ΔE′^{HM}| and calculating the number of mispaired base-pairs (n) by using the formula ΔΔE′˜n(Tₘ−Tₐ)ΔS, wherein Tₘ is the temperature at which half of the hybridized complex dissociates; wherein Tₐ is the temperature at which the nucleic acid probe is hybridized to the target nucleic acid; and wherein ΔS is the entropy of a single broken hydrogen bond.

In some embodiments, hybridizing the nucleic acid probe with the at least one target nucleic acid takes place in a buffer medium, wherein the buffer medium is water or any buffer allowing a hybridization of the nucleic acid molecules. In some embodiments, the buffer medium comprises: a) a salt selected from sodium chloride (NaCl), sodium citrate, and sodium phosphate, or any combination thereof; b) a detergent selected from the group consisting of sodium dodecyl sulfate (SDS), polyethylene glycol tert-octylphenyl ether (Triton X-100), nonylphenoxypoly(ethyleneoxy) ethanol (Nonidet P-40 (NP-40)), octylphenoxypolyethoxyethanol (Igepal CA-630), Polysorbate 20 (Tween 20), 3-[(3-cholamidopropyl)dimethylammonio]-1-propanesulfonate (CHAPS), N-lauroylsarcosine sodium salt (Sarkosyl) and sodium deoxycholate (Deoxycholate), or any combination thereof; c) a blocking agent selected from Denhardt's solution (comprising Ficoll, polyvinylpyrrolidone, and bovine serum albumin), salmon sperm DNA and herring sperm DNA, or any combination thereof; d) a buffer such as Tris-HCl-buffer, HEPES-NaOH, saline-sodium citrate buffer, saline-sodium phosphate-EDTA buffer, or any combination thereof; e) a chelating agent such as EDTA; and has a pH from 7.0 to 8.0. In some embodiments, the buffer medium is selected from the group consisting of standard SSC (Saline-Sodium Citrate) buffer, Denhardt's solution, Church buffer, formamide hybridization buffer, DIG Easy Hyb, HEPES buffer, and a lysis-buffer, as well as combinations thereof.

In some embodiments, the target nucleic acid is selected from the group consisting of DNA, RNA, PNA, XNA, LNA, morpholino oligomers, 2′O-Methyl-RNA, phosphorothioate DNA or RNA, guanosine-rich oligonucleotides, and D-amino acid-based nucleic acids, as well as combinations thereof.

In some embodiments, the fluorescent dye is selected from the group consisting of fluorescent dyes such as DAPI, ethidium bromide, SYBR Green, Alexa Fluor dyes, and CyDyes, Quantum Dots and lanthanide-based labels, as well as combinations thereof.

In some embodiments, the chaotropic agent selected from guanidinium chloride (GuHCl), guanidinium thiocyanate (GuSCN), phenol, formamide, urea, potassium ions (K′), lithium ions (Li⁺), and magnesium ions (Mg²⁺), as well as any combination thereof.

In some embodiments, the nucleic acid probe is excited by light in the wavelength corresponding to the absorption wavelength of the fluorescent dye. In some embodiments, the light is provided by a light-source selected from the group consisting of a mercury arc lamp, a xenon arc lamp, an LED, a LASER, a metal halide lamp, a tungsten-halogen lamp, and a deuterium lamp, as well as combinations thereof.

In some embodiments, the mispaired base pair is selected from the group consisting of A-A, A-C, C-A, A-G, G-A, T-T, T-C, C-T, T-G, G-T, C-C, G-G, U-U, A-U, U-A, as well as combinations thereof, wherein A is adenosine, C is cytosine, T is tyrosine, G is guanine, and U is uracil.

In some embodiments, the biological sample is a cell-lysate, a biological sample such as a tissue sample, or a body fluid selected from the group consisting of blood, urine, saliva, sweat, lymph fluid, cerebrospinal fluid (CSF), gastric juice, pleural fluid, peritoneal fluid, synovial fluid (joint fluid), and sputum (phlegm), as well as combinations thereof. In some embodiments, the method is for use in diagnostics outside the human or animal body or in medicine.

In some embodiments, the target nucleic acid is a nucleic target for use in the detection of a nucleic acid mispairing in diagnostics, research, or a therapeutic application in a biological and/or medical field.

In some aspects, the disclosure further relates to a method for analyzing a target nucleic acid in a biological sample in the presence of a sample molecule selected from a small molecule, peptide or protein comprising: a) contacting the biological sample comprising the sample molecule with a nucleic acid probe, wherein the nucleic acid probe is labelled with a fluorescent dye and adapted to hybridize with the target nucleic acid; b) hybridizing the nucleic acid probe and the target nucleic acid to produce a hybridized complex; c) irradiating the hybridized complex with an excitation light to excite the fluorescent dye; d) observing, with a detector, an emission of light from the fluorescent dye, wherein the emission of light has an energy of peak fluorescence (E^{P}); e) determining if the hybridized complex comprises an interference from the presence of the sample molecule, comprising: i) calculating ΔE and ΔE^{P}, wherein ΔE=E^{K}−E⁰ and ΔE^{P}=E^{P}−E^{0P}, wherein E^{0P} is a known energy of peak fluorescence of the nucleic acid probe in the presence of the sample molecule; wherein E⁰ is a known energy of peak fluorescence of the nucleic acid probe without the sample molecule; wherein E^{K} is an energy of peak fluorescence of the nucleic acid probe hybridized to a control nucleic acid having 100% complementarity to the nucleic acid probe without the sample molecule; ii) comparing |ΔE| and |ΔE^{P}|, wherein if |ΔE|≠|ΔE^{P}|, the hybridized complex comprises an interference from the sample molecule.

In some embodiments, the interference is selected from the group consisting of bending of the nucleic acid, looping of the nucleic acid, altering accessibility and compaction of the nucleic acid, unwinding of the nucleic acid, supercoiling the nucleic acid, and modify the nucleic acid, as well as combinations thereof.

In some aspects, the disclosure further relates to a method for detecting a drug-resistant tuberculosis infection in a subject, comprising steps of: a) obtaining a biological sample from a subject; b) contacting the biological sample with a nucleic acid probe, wherein the nucleic acid probe is labelled with a fluorescent dye and adapted to hybridize with at least one target nucleic acid; c) hybridizing the nucleic acid probe and the target nucleic acid to produce a hybridized complex; d) irradiating the hybridized complex with an excitation light to excite the fluorescent dye; e) observing, with a detector, an emission of light from the fluorescent dye, wherein the emission of light has an energy of peak fluorescence (E^{S}); f) determining if the hybridized complex has one or more mispaired base pairs, comprising: i) calculating ΔE^{H} and ΔE^{HM}, wherein ΔE^{H}=E⁰−E^{K} and ΔE^{HM}=E⁰−E^{S}, wherein E⁰ is a known energy of peak fluorescence of the nucleic acid probe; wherein E^{K} is a known energy of peak fluorescence of the nucleic acid probe hybridized to a control nucleic acid having 100% complementarity to the nucleic acid probe; ii) comparing |ΔE^{H}| and |ΔE^{HM}|, wherein if |ΔE^{H}|>|ΔE^{HM}|, the hybridized complex comprises at least one mispaired base pair; wherein the presence of one or more mispaired base pairs in the hybridized complex is indicative of a drug-resistant tuberculosis infection in the subject.

In some embodiments, the least one target nucleic acid comprises at least one of codons 531, 526, 516, 511, and 533 of the rpoB gene of Mycobacterium tuberculosis.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the forgoing general description and the following detailed description are exemplary and explanatory only and are not restrictive of the invention as claimed.

### BRIEF DESCRIPTION OF THE DRAWINGS

FIG. 1—shows the energy measurements which are the energy-shift upon hybridization of the probe with a 100% complementary control.

FIG. 2—shows the energy measurements which are the energy-shift upon hybridization of the probe with a sample nucleic acid, which for example has one mismatch.

FIG. 3—shows the energy measurements which are the repetition of the measurement shown in FIG. 1 in the same buffer but comprising a chaotropic agent.

FIG. 4—shows the different energy-differences which can used in order to qualitatively define that in the sample nucleic acid exists a mismatch as compared to the control.

FIG. 5—shows the different energy-differences which can used in order to quantitatively define how many mismatches are present in the sample nucleic acid as compared to the control.

FIG. 6—shows the alternative method which may be used in order to measure the influence of a molecule, such as small molecule, peptide or protein, on the duplex formed of the at least two nucleic acids present in the buffer medium.

FIG. 7 is a diagram showing the energy shift upon the formation of a duplex structure, with corresponding representative graph.

FIG. 8—Shows a sample of a detection device which may be used in order to measure the energy-shifts in the sample. Wherein 1. light source (laser/LED/ . . . ), 2. excitation irradiation, 3. beam splitter, 4. Mirror, 5. micro-objective, 6. sample plate, 7. Sample, 8. xyz translation stage, 9. emission (fluorescence), 10. emission filter (fluorescence filter), 11. coupling lens, 12. spectrometer (detector).

FIGS. 9A-9B—show a real depiction of the energy-shifts in buffer (FIG. 9A) and in a chaotropic substance guanidinium thiocyanate (FIG. 9B). Molecular probe (SEQ ID No. 1) 5′-AGA CCA GAA GAT CAG GAA CTC TA-3′ (Rhodamine 6G at 5′), Target without polymorph nucleotides: (SEQ ID No. 2) 5′-TA GAG TTC CTG ATC TTC TGG TCT-3′, Target with polymorph nucleotide: (SEQ ID No. 3) 5′-TA GAG TTC CTG ATT TTC TGG TCT-3′.

FIGS. 10A-10E—show the energy levels in buffers with different concentrations of a chaotropic agent (urea). FIG. 10A shows this with urea 0 mM, FIG. 10B with urea 1 mM, FIG. 10 C with urea 2 mM, FIG. 10D with urea 3 mM, and FIG. 10E with urea 4 mM. The energy-levels of the mismatched duplex shift below the energy level of the 100% complementary control at high concentrations of the chaotropic agent.

FIG. 11—shows the energy shift in the absence or presence of a “influencing” molecule, in this case spermidine. The molecular probe: (SEQ ID No. 4) 5′-GTG AGT TTG GGG AAA AAA AAT AAA ATA AAA ATG GCT TTC C-3′+Rhodamine 6G at 5′; The target sequence: (SEQ ID No. 5) 5′-GGA AAG CCA TTT TTA TTT TAT TTT TTT TCC CCA AAC TCA C-3′; Spermidin 5 mM.

FIG. 12 shows an example of the results obtained from the method described herein with tuberculosis, described in Example 6. FIG. 12 is a graph showing the normalized spectra of samples with the molecular beacon (MB) and perfect match (PM) (depicted by line labeled “POSITIVE”) and with only MB (depicted by line labeled “NEGATIVE”), as well as the differential between them.

FIGS. 13A-13B show the difference in line positions between MB, PM with MB, single mismatch (SM) with MB, and double mismatch (DM) with MB. FIG. 13B is a blown-up region of FIG. 13A.

FIG. 14 shows a normalized fluorescence intensity spectra of rhodamine 6G dye attached to linear molecular probes (LMPs) as described herein and two differential spectra obtained by subtracting the normalized spectra of the perfectly matched duplex from the spectra of the scrambled and mismatched targets. The top half of FIG. 14 shows normalized fluorescence intensity spectra of rhodamine 6G dye attached to linear molecular probes (LMPs) measured in the presence of 2 mM urea under three conditions: with a scrambled target (top line in legend), hybridized with a perfectly matching target (middle line in legend), and hybridized with a target containing a single mismatched nucleotide (bottom line in legend). The two zoomed insets display the energy displacement of the LMP spectrum measured with the scrambled target and the mismatched target in 2 mM urea-containing buffer. The bottom half of FIG. 14 shows two differential spectra obtained by subtracting the normalized spectrum of the perfectly matched duplex from the spectra of the scrambled (top line in legend) and mismatched (bottom line in legend) targets. The color of each line corresponds to the direction of the spectral shift: blue indicates a shift to higher energy (blue shift), and red indicates a shift to lower energy (red shift).

FIG. 15 shows fitted emission energies of the linear molecular probe measured under three conditions: a) single stranded state with a scrambled target (left box), b) double-stranded state with a perfectly matching target (middle box), and c) double stranded state with a target containing a single mismatched nucleotide (right box). For the SNP duplex, the emission energies are distributed across two well resolved bands: one corresponding to samples without urea (gray points) and the other samples containing x mM urea (red points). The right axis displays a relative temperature scale, representing the thermal equivalent (in kelvins, K) of the energy change with respect to the reference energy level of the perfectly matched duplex.

FIG. 16 shows emission energy of the linear molecular probe as a function of urea concentrations, measured under the conditions of FIG. 15. Each data point is shown with its corresponding standard deviation. Shaded areas are provided to illustrate overall trends in energy change in increase of the urea concentration. However, for the double stranded state with mismatched nucleotide, the effect of energy reduction (red shift) is believed to have a binary character. The right side of FIG. 16 shows an energetic diagram of the LMP in single and double stranded states, where the double stranded state is formed with perfectly matching target and mismatched target (SNP is indicated by the nucleotide with a red rim). Disruption of the water structure by urea molecules results in lowering of the free energy of the mismatched duplex, making it energetically favorable even as the perfectly matched duplex-red shifted. Unpaired hydrogen bonds in the single-stranded and mismatched duplex states are shown in red.

### DETAILED DESCRIPTION OF THE DISCLOSURE

### A. Definitions

As used herein, “nucleic acid” or “polynucleic acid” refers to the order or sequence of nucleotides along a strand of nucleic acids. The nucleic acid sequence may be single-stranded or double-stranded or contain portions of both double-stranded and single-stranded sequences. The nucleic acid sequence may be composed of DNA, both genomic and cDNA, RNA or DNA/RNA hybrid.

As used herein, “macromolecule” refers to any large biomolecule including nucleic acids, peptides, proteins, carbohydrates or lipids.

As used herein, “peptide” refers to short chains of between two and fifty amino acids, linked by peptide bonds. Chains of fewer than ten or fifteen amino acids may also be called oligopeptides, and include dipeptides, tripeptides, and tetrapeptides.

As used herein, “protein” refers to large biomolecules that are comprised of one or more long chains of amino acid residues. Proteins perform a vast array of functions within organisms, including catalysing metabolic reactions, DNA replication, responding to stimuli, providing structure to cells and organisms, and transporting molecules from one location to another. Proteins differ from one another primarily in their sequence of amino acids, which is dictated by the nucleotide sequence of their genes, and which usually results in protein folding into a specific 3D structure that determines its activity.

As used herein, “carbohydrate” refers to a biomolecule consisting of carbon (C), hydrogen (H) and oxygen (O) atoms, usually with a hydrogen-oxygen atom ratio of 2:1 (as in water) and thus with the empirical formula Cₘ(H₂O)ₘ (where m may or may not be different from n). However, not all carbohydrates conform to this precise stoichiometric definition (e.g., uronic acids, deoxy-sugars such as fucose), nor are all chemicals that do conform to this definition automatically classified as carbohydrates (e.g. formaldehyde). It is used herein primarily as a synonym of saccharide, a group that includes sugars, starch, and cellulose.

As used herein, “lipid” refers to a macro biomolecule that is soluble in nonpolar solvents. Non-polar solvents are typically hydrocarbons used to dissolve other naturally occurring hydrocarbon lipid molecules that do not (or do not easily) dissolve in water, including fatty acids, waxes, sterols, fat-soluble vitamins (such as vitamins A, D, E, and K), monoglycerides, diglycerides, triglycerides, and phospholipids. As such the term encompasses fatty acids, glycerolipids, glycerophospholipids, sphingolipids, sterols, prenols and saccharolipids.

As used herein the term “sensor-probe” is used herein for any molecule which is able to specifically bind to a target molecule, i.e. via hybridization and/or other binding modes, such as for example via van-der-Waals-bonds, covalent bonds and/or hydrogen bonds. Thus, the term “sensor-probe” without further definition encompasses “quenched molecular probes”, “molecular beacons”, or “(molecular beacon) probes”, as well as “target-binding molecules” (such as for example antibodies).

As used herein, the terms “target/probe molecule complex” or “complex” are used in a general manner for any group of two or more associated molecules. For example, such an association may be the hybridization of the molecular beacon and the target nucleic acid and/or the binding of the target-binding molecule and the target macromolecule. The complex is excited by light and the emission is detected in the analysing device.

As used herein, “molecular beacons” are oligonucleotides that can report the presence of specific nucleic acids in homogenous solutions. Molecular beacons are hairpin-shaped molecules with a fluorochrome. This is a novel nonradioactive method for detecting specific sequences of nucleic acids. They are useful in situations where it is either not possible or desirable to isolate the molecular beacon-target hybrids from an excess of the molecular beacons. Molecular beacons are a particular case of molecular probes. In some embodiments of the invention, the molecular probes implemented are not molecular beacons, do not comprise a loop, and are >80 bp, as described herein. While a molecular beacon comprises a loop, a “quenched molecular probe” can be used to define probes that have a fluorophore and a quencher on opposite ends. Such probes may form a kind of stem (or the entire probe may function as a stem), but they may have no loop or several loops.

A typical molecular beacon is 25-40 nucleotides long. The middle nucleotides are complementary to the target DNA or RNA and do not base pair with one another, while the five to seven nucleotides at each terminus are complementary to each other rather than to the target DNA.

A typical molecular beacon structure can be divided in 4 parts: 1) loop, an 18-30 base pair region of the molecular beacon that is complementary to the target sequence; 2) stem formed by the attachment to both termini of the loop of two short (5 to 7 nucleotide residues) oligonucleotides that are complementary to each other; 3) 5′ fluorescent dye at the 5′ end of the molecular beacon, a fluorescent dye is covalently attached; 4) 3′ quencher (non-fluorescent) dye that is covalently attached to the 3′ end of the molecular beacon. When the beacon is in closed loop shape, the quencher resides in proximity to the fluorescent dye, which results in quenching the fluorescent emission of the latter. If the nucleic acid to be detected is complementary to the strand in the loop, the event of hybridization occurs. The duplex formed between the nucleic acid and the loop is more stable than that of the stem because the former duplex involves more base pairs. This causes the separation of the stem and hence of the fluorescent dye and the quencher. Once the fluorescent dye is no longer next to the quencher, illumination of the hybrid with light results in the fluorescent emission. The presence of the emission reports that the event of hybridization has occurred and hence the target nucleic acid sequence is present in the test sample. The fluorescent dye of the molecular beacon may be any suitable fluorescent dye, for example ABI dyes (e.g. FAM™, HEX™, TET™, JOE™, ROX™, CAL Fluor™ Red 610), cyanine dyes (e.g. Yakima Yellow or ATTO) or molecular dyes (e.g. ALEXA-fluor or BODIPY dyes). The quencher of the molecular beacon may be any suitable quencher, for example TAM, BHQ1, BHQ2, DAB, Eclip, BBQ650. Molecular beacons are synthetic oligonucleotides whose preparation is well documented in the literature (Tyagi S; Kramer F R, (1996). “Molecular beacons: probes that fluoresce upon hybridization”. Nat. Biotechnol. 14(3): 303-308). Further, the molecular beacon design is within the skill of a molecular biologist and there are many bioinformatics tools for this purpose available (e.g. Beacon Designer™). In addition, molecular beacons are commercially available for many target sequences (e.g. Eurofins Genomics, Ebersberg, Germany; Integrated DNA Technologies, Inc., Coralville, Iowa, USA).

In the context of this disclosure, “hybridization” means hydrogen bonding, which may be Watson-Crick, Hoogsteen or reversed Hoogsteen hydrogen bonding, between complementary nucleoside and/or nucleotide bases. Hybridization takes place under stringent or non-stringent conditions. As used herein, “stringent” refers to the conditions, i.e. temperature, buffer composition or ionic strength under which hybridization between polynucleotides occurs. These conditions depend mainly on the composition and complexity of the target nucleic acid and length of the molecular beacon probe.

For the hybridization temperature conditions the “Tₘ” (melting temperature) of the nucleic acids has to be considered. “Tₘ” means under specified conditions the temperature at which half of the nucleic acid sequences are disassociated and half are associated. Generally, suitable hybridization conditions may be easily determined by a person skilled in the art.

“Complementary” as used herein, refers to the capacity for pairing between two nucleotides. For example, if a nucleotide at a certain position of an oligonucleotide is capable of hydrogen bonding with a nucleotide at the same position of a DNA or RNA molecule, then the oligonucleotide and the target DNA or RNA are considered to be complementary to each other at that position. For example, the sequence 5′-A-C-T-3′ is complementary to the sequence 3′-T-G-A-5′.

Complementarity may be partial, in which only some of the nucleotides match according to base pairing, or complete, where all the nucleotides match according to base pairing. For purposes of the present disclosure “substantially complementary” refers to 90% or greater identity over the length of the target base pair region. The complementarity can also be 45, 50, 60, 70, 75, 80, 85, 90, 91, 92, 93, 94, 95, 96, 97, 98, 99, or 100% complementary, or any amount below or in between these amounts. In other words, the oligonucleotide and the target DNA or RNA are complementary to each other when a sufficient number of corresponding positions in each molecule are occupied by nucleotides which can build a hydrogen bond with each other. Thus, “specifically hybridizable” and “complementary” are terms which are used to indicate a sufficient degree of complementarity or precise pairing such that stable and specific binding occurs between the molecular beacon probe and the DNA or RNA target. It is understood in the art that the molecular beacon probe does not need to be 100% complementary to that of its target nucleic acid to be specifically hybridizable. “Oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly.

As used herein, “target-binding molecule” refers to a molecule, preferably a protein, which is able to specifically bind to a target macromolecule (such as a peptide, protein, carbohydrate or lipid) in a sample. Such a target-binding molecule may be selected from the group comprising antibody, antibody-fragment, Fab-fragment, anticalin protein, DARPin, nanoCLAMP, affilin, affimer, affitin, alphabody, avimer, Kunitz domain peptide, monobody, intrabody, microantibody, single-chain variable fragment (scFv), and combinations thereof. Target-binding molecules typically bind to unique surface patterns on the target macromolecule, sometimes referred to as “epitope”.

As used herein, “specifically binding” refers to the binding of the target-binding molecule to the target macromolecule (such as a peptide, protein, carbohydrate or lipid) in a sample with a high specificity. The fewer ligands a protein can bind besides the desired target, the greater is its specificity. Specificity describes the strength of binding between a given protein and ligand. This relationship can be described by a dissociation constant (K_{D})), which characterizes the balance between bound and unbound states for the protein-ligand system. K_{D} is the equilibrium dissociation constant, a calculated ratio of K_{off}/Kₒₙ, between the target-binding molecule and its target. The association constant (Kₒₙ) is used to characterise how quickly the target-binding molecule binds to its target. The dissociation constant (K_{off}) is used to measure how quickly a target-binding molecule dissociates from its target. The specificity of the target-binding molecule is at least micromolar (μM, K_{D} between 10⁻⁴ to 10⁻⁶), more preferably nanomolar (nM, K_{D} between 10⁻⁷ to 10⁻⁹), even more preferably picomolar (pM, K_{D} between 10⁻¹⁰ to 10⁻¹²), preferably femtomolar (fM, K_{D} between 10⁻¹³ to 10⁻¹⁵), and most preferably attomolar (fM, K_{D} between 10⁻¹⁶ to 10⁻¹⁸).

“Subject” or “patient” as used herein refers to a living organism, such as a mammalian individual, including murines, cattle, simians and humans. Preferably, the patient is a human.

“Biological sample” or just “sample” as used herein refers to a biological sample encompassing liquid and solid samples. Liquid samples encompass saliva, sputum, sweat, urine, nasal secretion, bronchoalveolar lavage fluid, laryngo-pharyngeal scrape test, vaginal secretion, blood liquids (e.g. serum, plasma) and cerebrospinal fluid (CSF). Solid samples encompass tissue samples such as tissue cultures or biopsy specimen. A preferred patient's sample is sputum. The patient's sample will be collected with the disposable pipette as described below.

In a particular preferred embodiment of the present disclosure, the method is used for detecting a biomarker (including pathogens) in a patient's sample.

“Biomarker” as used herein, is a measurable indicator of some biological state or condition. Biomarkers are often measured and evaluated using blood, urine, or soft tissues to examine normal biological processes, pathogenic processes, or pharmacologic responses to a therapeutic intervention. As such the disclosure pertains to labeling a biomarker such as a macromolecule (such as nucleic acids, peptides, proteins, carbohydrates or lipids) contained in a patient's sample (e.g. blood-sample, liquor-sample and/or saliva sample comprising the target macromolecule) with a sensor-probe. For simplification the definition of “biomarker” within this application encompasses also pathogens.

“Pathogens” as used herein, is any organism that can produce disease. A pathogen may also be referred to as an infectious agent, or simply a germ. Typically, the term is used to describe an infectious microorganism or agent, such as a virus, bacterium, protozoan, prion, viroid, or fungus.

As used herein, “nucleic acid” or “polynucleic acid” refers to the order or sequence of nucleotides along a strand of nucleic acids. The nucleic acid sequence may be single-stranded or double-stranded or contain portions of both double-stranded and single-stranded sequences. The nucleic acid sequence may be composed of DNA, both genomic and cDNA, RNA or DNA/RNA hybrid.

The fluorescent dye attached (optionally covalently bound) to the probe may be any suitable fluorescent dye, for example ABI dyes (e.g. FAM™, HEX™, TET™, JOE™, ROX™, CAL Fluor™ Red 610), cyanine dyes (e.g. Yakima Yellow or ATTO) or molecular dyes (e.g. ALEXA-fluor or BODIPY dyes). The quencher of the probe may be any suitable quencher, for example TAM, BHQ1, BHQ2, DAB, Eclip, BBQ650.

In the context of this disclosure, “hybridization” means hydrogen bonding, which may be Watson-Crick, Hoogsteen or reversed Hoogsteen hydrogen bonding, between complementary nucleoside or nucleotide bases. Hybridization takes place under stringent or non-stringent conditions.

As used herein, “stringent” refers to the conditions, i.e. temperature, buffer composition or ionic strength under which hybridization between polynucleotides occurs. These conditions depend mainly on the composition and complexity of the target nucleic acid and length of the probe.

For the hybridization temperature conditions the “Tm” (melting temperature) of the nucleic acids has to be considered. “Tm” means under specified conditions the temperature at which half of the nucleic acid sequences are disassociated and half are associated. Generally, suitable hybridization conditions may be easily determined by a person skilled in the art.

“Complementary” as used herein, refers to the capacity for pairing between two nucleotides. For example, if a nucleotide at a certain position of an oligonucleotide is capable of hydrogen bonding with a nucleotide at the same position of a DNA or RNA molecule, then the oligonucleotide and the target DNA or RNA are considered to be complementary to each other at that position.

For example, the sequence 5′-A-C-T-3′ is complementary to the sequence 3′-TG-A-5′. Complementarity may be partial, in which only some of the nucleotides match according to base pairing, or complete, where all the nucleotides match according to base pairing. For purposes of the present disclosure “mispairing” refers to a situation where at least two bases are not complementary to each other, e.g. where the base-pair is C-A, A-C, T-G, G-T, U-A or A-U.

The term “substantially complementary” refers to 90% or greater identity over the length of the target base pair region. The complementarity can also be 45, 50, 60, 70, 75, 80, 85, 90, 91, 92, 93, 94, 95, 96, 97, 98, 99, or 100% complementary, or any amount below or in between these amounts. In other words, the oligonucleotide and the target DNA or RNA are complementary to each other when a sufficient number of corresponding positions in each molecule are occupied by nucleotides which can build a hydrogen bond with each other. Thus, “specifically hybridizable” and “complementary” are terms which are used to indicate a sufficient degree of complementarity or precise pairing such that stable and specific binding occurs between the probe and the DNA or RNA target. It is understood in the art that the probe does not need to be 100% complementary to that of its target nucleic acid to be specifically hybridizable.

“Oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly.

“Subject” or “patient” as used herein refers to a mammalian individual, including murines, cattle, simians and humans. Preferably, the patient is a human.

“Sample” as used herein refers to a biological sample encompassing liquid and solid samples. Liquid samples encompass saliva, sputum, sweat, urine, nasal secretion, bronchoalveolar lavage fluid, laryngo-pharyngeal scrape test, vaginal secretion, blood liquids (e.g. serum, plasma) and cerebrospinal fluid (CSF). Solid samples encompass tissue samples such as tissue cultures or biopsy specimen. A preferred patient's sample is sputum. The patient's sample will be collected with a disposable device as described hereinunder.

“Treatment,” as used herein refers to an intervention performed with the intention of preventing the development or altering the pathology of a disorder. Accordingly, “treatment” refers to both therapeutic and prophylactic or preventative measures. Those in need of treatment include those already with the disorder as well as those in which the disorder is to be prevented. Standard treatment for TB currently comprises a standard regimen of rifampin in combination with isoniazid, ethambutol and pyrazinamide for the treatment of drug-susceptible TB, or second line antibiotics for drug-resistant TB. A treatment for drug-resistant or rfampin-resistant tuberculosis should only include drugs to which the subject's tuberculosis is susceptible. For example, isonaizid-resistant tuberculosis treatment comprises a six-month daily regimen of rifampin, ethambutol, pyrazinamide, and a later-generation fluoroquinoline. Rifampin-resistant or multidrug-resistant tuberculoisis treatment comprises, in some embodiments, a six-month treatment regimen of bedaquiline, pretomanid, linezolid, and moxifloxacin. For subjects with fluoroquinoline resistance or intolerance, treatment comprises a six-month treatment region of bedaquiline, pretomanid, and linezolid.

In a particular preferred embodiment of the present disclosure, the method is used for detecting a virus in a patient's sample. “Virus” or “viral” means any single- or double-stranded DNA or RNA virus. In particular, Human Immunodeficiency Virus (HIV), Zika, MERS-Corona, SARS-COV-1, Sars-COV-2, Hepatitis B Virus (HBV), Hepatitis C Virus (HCV), Human Lyphotropic Virus-type 1-4 (HTLV-1-4), Epstein-Barr Virus (EBV), Human Papilloma Virus (HPV), Influenza A, B or C, Non-Influenza Respiratory Viruses (NIRVs, e.g. respiratory syncytial virus, parainfluenza virus, rhinovirus, metapneumovirus), norovirus, Ebola virus, Reovirus, Herpes virus (simplex 1, 2+B, 5, 6, 7, 8), Pox virus, FSME virus, Dengue virus, Rubivirus, Varizella zoster virus and Cytomegalievirus.

In a method according to the disclosure, the target nucleic acid may in one embodiment be a virus RNA. This method provides for screening large numbers of patients for infection, in an extremely reduced time scale, compared to the state of the art. The virus may in particular be a coronavirus, further in particular SARS-COV-2. The method is thus applicable in a recent world-wide pandemic situation. In a method according to the disclosure, the excitation light may in particular be a laser beam. This method allows for limiting the excitation light to the excitation wavelength of the fluorescent dye, and thus for avoiding side effects induced by other wavelengths.

Types of nucleic acids disclosed hereinunder:

Genomic DNA: Detecting specific genes, mutations, or overall genome integrity.

Mitochondrial DNA (mtDNA): Used for studying maternal lineage, certain diseases, and forensic analysis.

Plasmid DNA: Common in bacterial studies and biotechnology applications.

Viral DNA: Detection of DNA viruses like herpesvirus, human papillomavirus (HPV), and adenovirus.

mRNA (messenger RNA): Reflects gene expression levels; used in gene expression studies and diagnostics.

rRNA (ribosomal RNA): Important for detecting and quantifying ribosomes; used in microbial identification and taxonomy.

tRNA (transfer RNA): Less common in diagnostics but crucial for understanding translation processes.

miRNA (microRNA): Small non-coding RNA molecules involved in gene regulation; used in cancer and other disease diagnostics.

siRNA (small interfering RNA): Used in gene silencing studies and therapeutic applications.

Viral RNA: Detection of RNA viruses like influenza, HIV, SARS-COV-2 (the virus responsible for COVID-19), hepatitis C virus (HCV), and Zika virus.

lncRNA (long non-coding RNA): Involved in gene regulation; studied in various diseases including cancer.

snRNA (small nuclear RNA): Involved in RNA splicing; studied in certain genetic disorders.

### B. Embodiments of the Invention

As outlined before, in one embodiment the disclosure relates to a method for detecting nucleic acid hybridization, comprising the steps of:
a) measuring the energy (E⁰) of a probe in a buffer, comprising the steps of
   a1) providing a buffer medium;
   a2) providing a nucleic acid probe with a fluorescence-label within that buffer medium;
   a3) measuring the energy (E⁰) of that system by exciting the fluorescence-tag and measuring the fluorescence of the system;
b) measuring the energy (E^{K}) of the probe hybridized to a control nucleic acid, comprising the steps of:
   b1) using the same buffer medium of step a);
   b2) providing the probe of step a2) and a control nucleic acid molecule which is 100% complementary to the nucleic acid probe;
   b3) letting the probe and the complementary nucleic acid molecule hybridize at a temperature Ta which is from 0° C. to the melting temperature of both molecules, preferably from above 0° C. and up to 5° C. below the melting temperature of both molecules;
   b4) measuring the energy (E^{K}) of that system by exciting the fluorescence-tag and measuring the fluorescence of the system;
c) measuring the energy (E^{S}) of the probe hybridized to a sample nucleic acid, comprising the steps of:
   c1) using the same buffer medium of steps a) and b);
   c2) providing the probe of steps a2) and b2) and a sample nucleic acid molecule;
   c3) letting the probe and the sample nucleic acid molecule hybridize at the same temperature Ta as used in step b3);
   c4) measuring the energy (E^{S}) of that system by exciting the fluorescence-tag and measuring the fluorescence of the system;
and optionally,
d) repeating steps a)-c) in a buffer with a chaotropic agent; thereby receiving the energies E′⁰, E′^{K} and E′^{S}.
e) calculate the energy-shifts, comprising the steps of:
   e1) calculate ΔE^{H} according to the formula ΔE^{H}=E⁰−E^{K};
   e2) calculate ΔE^{HM} according to the formula ΔE^{HM}=E⁰−E^{S}.
   e3) calculate ΔE′^{H} according to the formula ΔE′^{H}=E′⁰−E′^{K};
   e4) calculate ΔE′^{HM} according to the formula ΔE′^{HM}=E′⁰−E′^{S}.
   e5) calculate ΔΔE according to the formula ΔΔE=|ΔE^{H}−|ΔE^{HM};
e6) optionally, calculate ΔΔE according to the formula ΔΔE′=|ΔE′^{HM}|−|ΔE′^{H}|;
wherein |ΔE^{H}|>|ΔE^{HM}|—means that there is at least a single mispaired base-pair in the duplex of probe and sample nucleic acid;
wherein ΔΔE means a decrease of the duplex melting temperature;
wherein, optionally, |ΔE′^{H}|<|ΔE′^{HM}| means that in the duplex are mispaired base-pairs and
wherein ΔΔE′ can be used to calculate the number of mispaired base-pairs by using the
formula ΔΔE′˜n (Tm−Ta)ΔS, where n is the number of mispaired base pairs, Tₘ—is the melting temperature, Tₐ is the temperature as of step b3), and ΔS is the entropy of single broken hydrogen bond.

Wherein the energy shift of a mispairing event can be estimated from nearest neighbour theory (SantaLucia J (1998) A unified view of polymer, dumbbell, and oligonucleotide DNA nearest-neighbor-thermodynamics, Proceedings of the National Academy of Sciences 95(4):1460-1465).

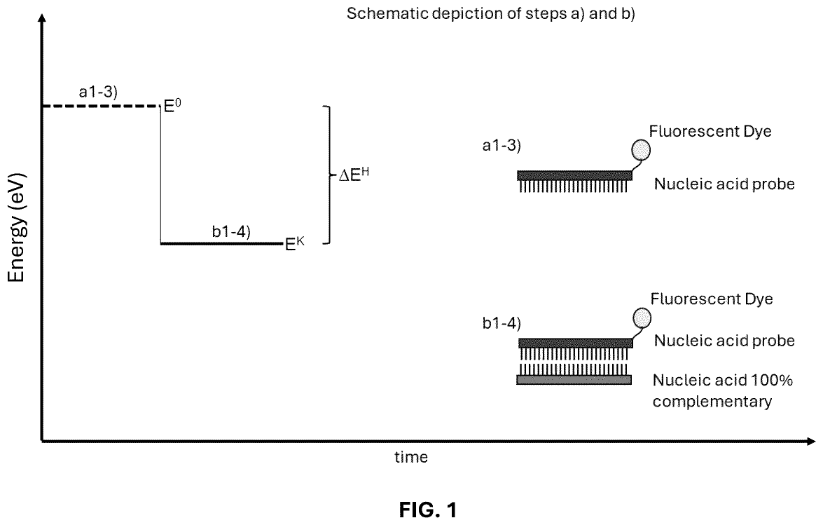

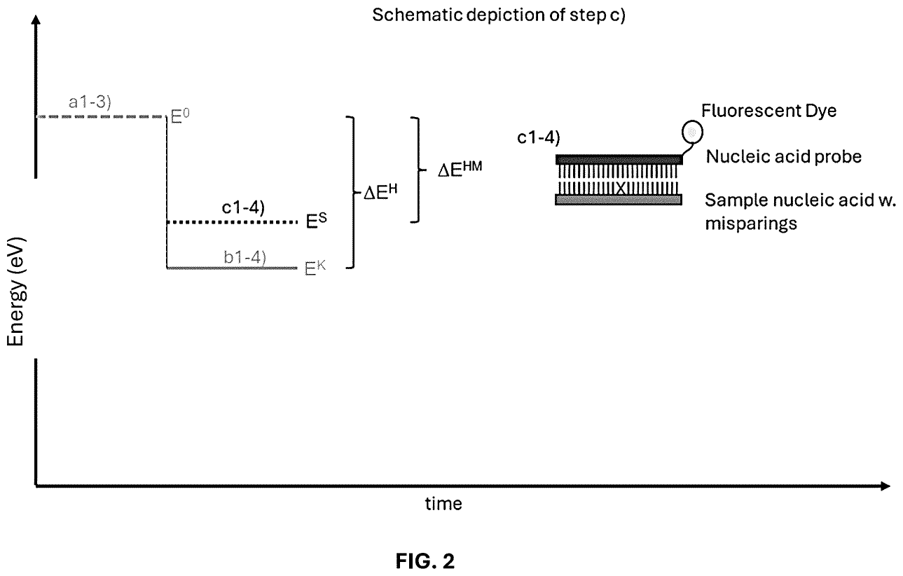

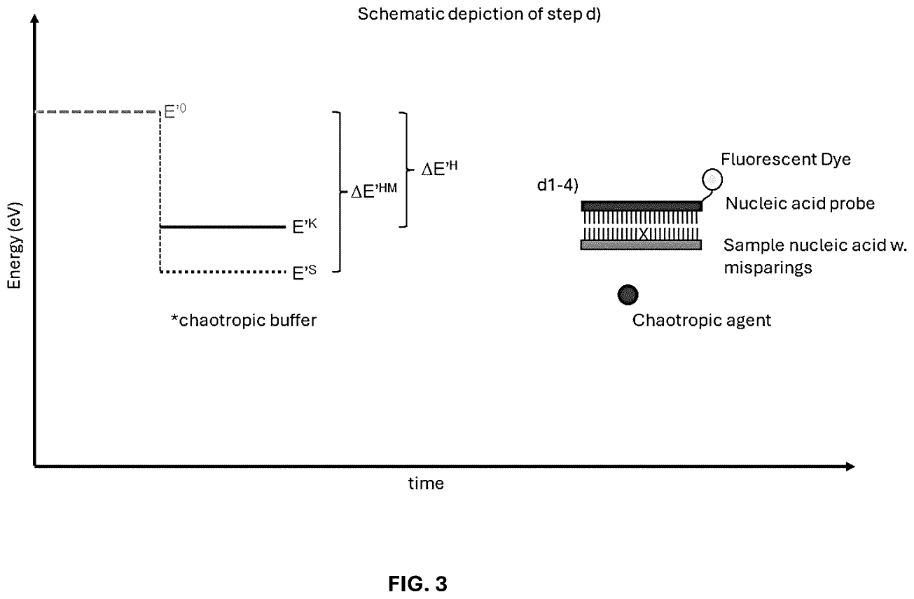

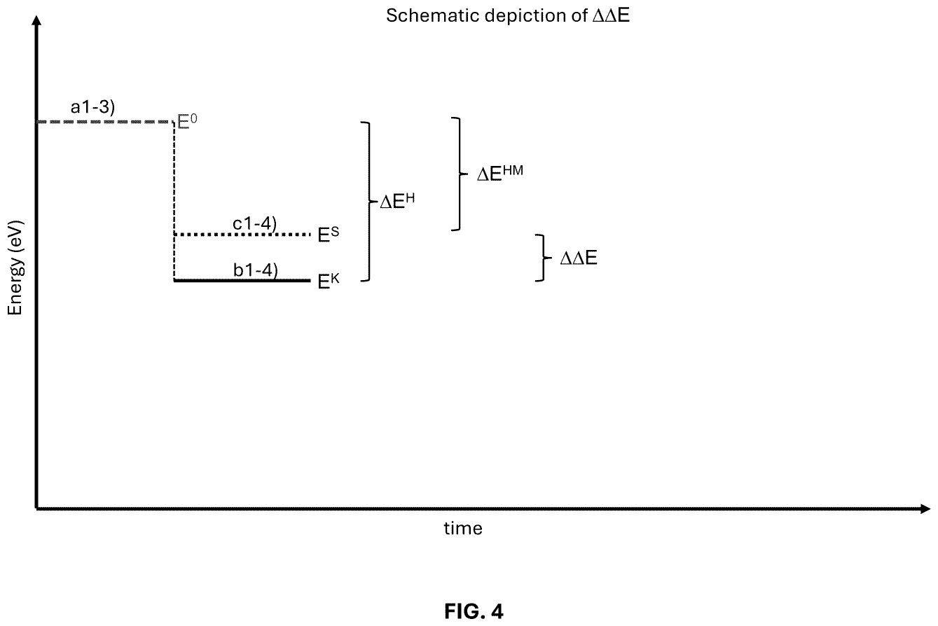

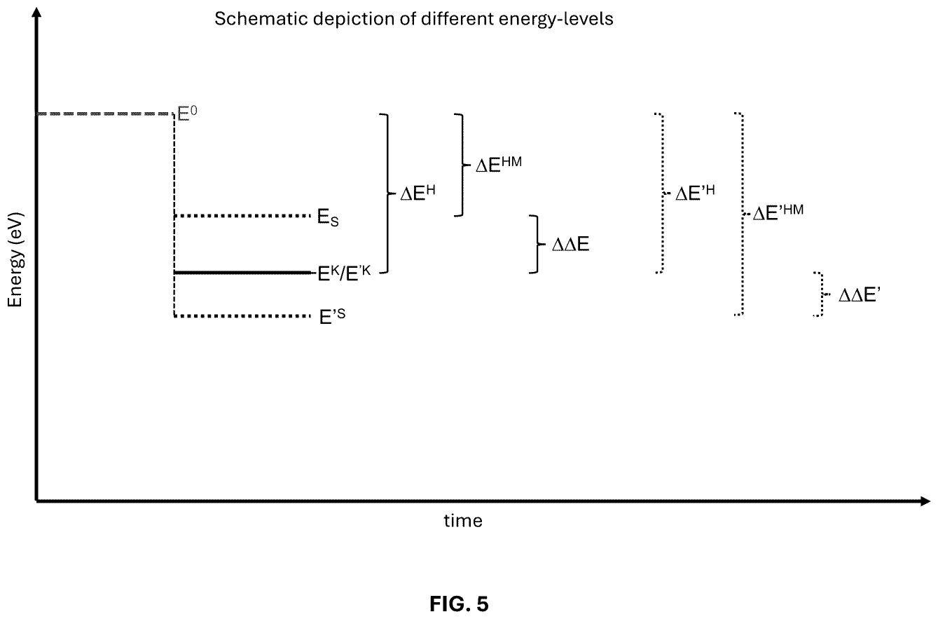

Please be also referred to FIGS. 1 to 5 which explain the method in further detail.

The following paragraphs present a summary of the methods described herein: In general terms, to visualize the effect of hybridization, the following steps are carried out. First, the spectrum without the target in the solution (negative) is measured. Then, the spectrum with the target in solution (positive) is measured. Both spectra are normalized to the same value, either to 1 or to the total amount of photons. These values are subtracted from one another (spectrum with target minus spectrum without target). The resulting spectrum shows a shift toward higher or lower energies (blue or red shift, respectively), if hybridization is observed, or a flat noisy line if no hybridization occurs.

In general, to analyze the data obtained, the following steps are carried out. First, the spectra for a sample without the target (negative) is measured, and the line position is determined. Then, the spectra for a sample with the target (positive) is measured, and the line position is determined. The difference in line positions between negative and positive corresponds to the hybridization energy. If the target contains at least one single mismatched nucleotide and the hybridization buffer includes a chaotropic agent, then the hybridization energy will be approximately 0.9 meV (or 10 K) larger (stronger) compared to the target without such mismatch (perfect match target). In other words, a variant (e.g., Rifampicin resistance) can be identified simply by observing that the hybridization energy is about 0.9 meV (10 K) higher than the expected hybridization energy.

The optical method as disclosed hereinunder allows for quantitative measurements of nucleic acid hybridization and mutations at room temperature could offer several significant advantages:

### Simplicity and Speed

Rapid Results: Potential for faster analysis compared to traditional methods.

User-Friendly: Likely easier to use with minimal sample preparation.

### Cost-Effectiveness

Lower Costs: Reduced need for expensive reagents, consumables, and equipment.

Minimal Infrastructure: Less requirement for specialized lab infrastructure.

Qualitative and, optionally, quantitative Analysis

Precise Measurement: Provides quantitative data on the amount of hybridized nucleic acid.

Real-Time Monitoring: Ability to monitor hybridization events in real-time.

### Room Temperature Operation

Convenience: Simplifies experimental setup and reduces the need for temperature control.

Stability: Reduces potential for thermal degradation of samples or reagents.

### High Throughput and Scalability

Parallel Processing: Potential for high-throughput analysis of multiple samples simultaneously.

Scalability: Easily scalable to accommodate varying sample sizes and experimental demands.

### Reduced Risk of Artifacts

Minimized Interference: Lower risk of artifacts from complex sample preparation steps.

Direct Detection: Potential for direct detection of nucleic acid interactions without extensive amplification or labeling.

The term “probe” is used herein for a single-stranded piece of nucleic acid, such as DNA or RNA, that is designed to be complementary to a specific sequence of nucleic acids in a target molecule, wherein the probe is no longer than the target, or the same length as the range of interest. The primary function of these probes is to detect the presence of the target sequence in a sample, such as a sample nucleic acid, by hybridizing (binding) to it through complementary base pairing. The nucleic acid probes of the present disclosure are labelled with a fluorescent molecule, they may also be referred to as fluorescent probes. The fluorescent label is a molecule that can absorb light at a particular wavelength and then emit light at a different, usually longer, wavelength. This property allows the probe to be detected and measured using fluorescence detection systems, which are highly sensitive and can provide quantitative data.

The fluorescent label is typically attached to one end of the nucleic acid probe or incorporated into its structure. When the probe binds to its target sequence, the fluorescence can be detected, indicating the presence of the target nucleic acid in the sample. Fluorescent labelling is advantageous because it allows for real-time monitoring and can provide a high degree of specificity and sensitivity.

In one embodiment the probe is a quenched molecular probe. That is a specific type of fluorescently labelled nucleic acid probe. It has a unique structure that allows it to fluoresce only when it is bound to its target sequence. This property makes molecular beacons particularly useful for detecting specific nucleic acid sequences in complex mixtures or for monitoring dynamic biological processes.

A molecular beacon may consist of three main parts:
Loop Region: Single stranded sequence which is complementary to the target nucleic acid sequence. In the disclosed invention, it is essential to have entire sequence complementary to the target.
Stem Region: Flanking complementary sequences that form a double-stranded stem. This stem keeps the probe closed so that the dye is quenched with the quencher The stem must be complementary to the target nucleic acid.
Fluorophore and Quencher: At opposite ends of the stem, a fluorophore and a quencher are attached. The quencher is a molecule that absorbs the fluorescence emitted by the fluorophore when they are in close proximity, thereby preventing fluorescence.

In the absence of the target sequence, the quenched molecular probe remains quenched, bringing the fluorophore and quencher close together. This proximity allows the quencher to suppress the fluorescence of the fluorophore, making the probe non-fluorescent.

When the quenched molecular probe encounters its target sequence, the complementary base pairing between the loop and the target sequence is stronger than the base pairing in the stem. This causes the stem to unwind and the loop to hybridize with the target. As a result, the fluorophore and quencher are separated, allowing the fluorophore to emit fluorescence. The intensity of the fluorescence is directly proportional to the amount of target sequence present, making molecular beacons useful for quantitative assays.

The typical length of the nucleic acid probe, including those used as molecular beacons, may range from 5 to >100 nucleotides, from 5 to >80 nucleotides, from 5 to 80 nucleotides 5 to 50 nucleotides, from 8 to 45 nucleotides, from 10 to 40 nucleotides, from 15 to 30 nucleotides, from 18 to 25 nucleotides or about 20 nucleotides. In some embodiments, the length of the nucleic acid probes used herein is greater than 80 bp. In some embodiments, this length is limited only by technological limitations in oligonucleotide manufacturing, as one skilled in the art will understand. This length is generally sufficient to provide specific and stable hybridization with the target sequence while avoiding non-specific binding.

In some embodiments, the nucleic acid probes described herein are not molecular beacons. In some embodiments, the nucleic acid probes described herein are segments of real DNA labelled with dye and a respective quencher. In some embodiments, these nucleic acid probes comprise a length of greater than 80 bp. In some embodiments, the sequences of the nucleic acid probes described herein are selected such that it comprises the desired codon, sequence, mutation, or any other desired factor, and such that folding most probably will quench the dye.

The design of the probe may take into account the following factors:

The sequence of the probe is carefully selected to be complementary to a unique region of the target nucleic acid, minimizing the likelihood of cross-reactivity with other sequences. In some embodiments, the sequence of the probe is selected to be complementary to the specific region of the target. In some embodiments, specificity is achieved using extra long nucleic acid probes. In some such embodiments, the extra long nucleic acid probes are extra long molecular beacons.

The length of the probe also influences its melting temperature (Tm), which is the temperature at which half of the probe-target duplex dissociates.

In one embodiment the hybridization done in steps b3), c3) and d3), as well as steps cc3) and dd3), at an temperature (Ta) which is below Tm (Ta<Tm). In some embodiments Ta is a temperature from 0° C. to the melting temperature of both molecules. In some embodiments Ta is below Tm of at least 1° C., of at least 2° C., of at least 3° C., of at least 4° C., or of at least 5° C. Ta is in one embodiment at 0° C., in other embodiments above 0° C. (Ta>0° C.) in order to avoid freezing of the buffer medium. It depends on the freezing point of the buffer medium how low Ta may be selected. Thus, in one embodiment Ta is >freezing point of the buffer medium. In further embodiments Ta is 1° C. or more, 2° C. or more, 3° C. or more, 5° C. or more, 10° C. or more, or 15° C. or more. Thus, in one embodiment Ta may be selected from freezing point of buffer to Tm, from >freezing point of buffer to <Tm, 0° to Tm, from 0° C. to <Tm, from >0° C. to <Tm, from 0° C. to Tm−1° C., from 0° C. to Tm−2° C., from 0° C. to Tm−3° C., from 0° C. to Tm−4° C., from 0° C. to Tm−5° C., from >0° C. to Tm−1° C., from >0° C. to Tm−2° C., from >0° C. to Tm−3° C., from >0° C. to Tm−4° C., from >0° C. to Tm−5° C., from 1° C. to Tm−1° C., from 2° C. to Tm−2° C., from 3° C. to Tm−3° C., from 5° C. to Tm−4° C., or from 10° C. to Tm−5° C.

The term “freezing point” is defined herein as the temperature at which a liquid turns into a solid under normal atmospheric pressure. At this temperature, the molecules of the liquid slow down enough due to a decrease in thermal energy to form a solid crystalline structure. This phase transition is a characteristic property of the substance and varies depending on the nature of the liquid.

In a further embodiment a method is disclosed wherein instead to the described steps c) to e), steps cc) and dd) and ee) are performed, comprising:
cc) repeating step b) in the presence of a sample molecule selected from a small molecule, peptide or protein, thereby measuring the energy E^{P} which is the energy of the hybridized nucleic acid molecule in the presence of the sample molecule;
dd) calculate ΔE according to the formula ΔE=E^{K}−E⁰; calculate ΔE′P according to the formula ΔE′=E′^{K}−E′⁰;
ee) calculate ΔE^{P} according to the formula ΔE^{P}=E^{P}−E^{0p}, calculate ΔE′^{P} according to the formula ΔE′^{P}=E′^{P}−E′^{0P},
wherein the difference between the values of ΔE and ΔE^{P} and ΔE′ and ΔE′^{P} indicates an interaction of the sample molecule with the duplex of the probe and the sample nucleic acid molecule.

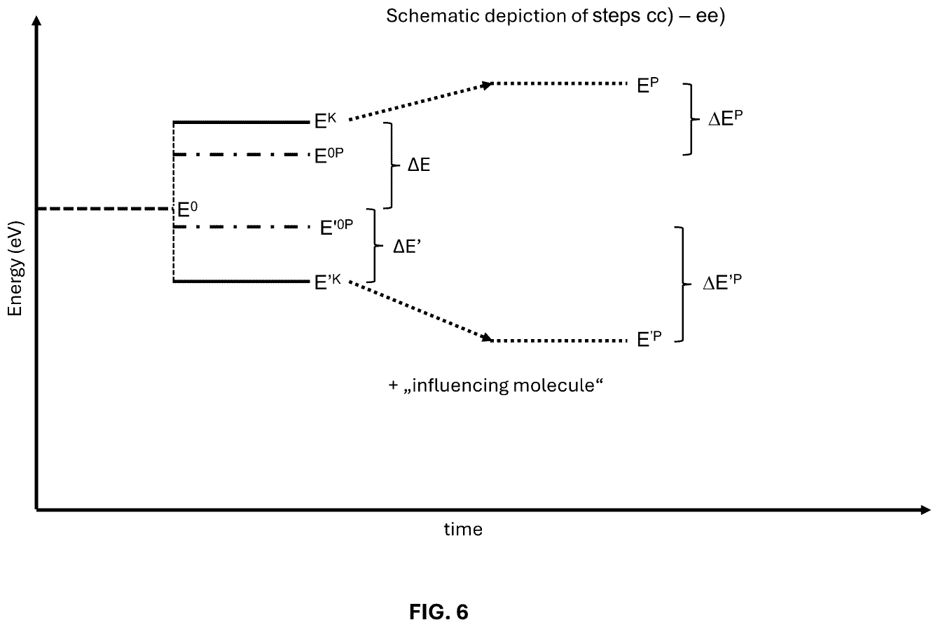

Please note also FIG. 6 for further information.

As outlined above the method may also be used to detect the “interference” of a molecule, selected from a small molecule (i.e. a small organic molecule), peptide and/or protein on the nucleic acid duplex. In such a case the molecule is added to the buffer and the energy shifts with respect to the solutions without the presence of the molecule are detected and calculated.

In another embodiment of the disclosure the “interference of the molecule” (such as a sample small molecule (i.e. a small organic molecule), peptide and/or protein) with the nucleic acid is selected from the group consisting of bending of the nucleic acid, looping of the nucleic acid, altering accessibility and compaction of the nucleic acid, unwinding of the nucleic acid, supercoiling the nucleic acid, and modify the nucleic acid, as well as combinations thereof.

Further, the method may also be used to define “permissive” buffer medium conditions. Permissive buffer conditions are conditions in which the energy difference between 100% complete hybridization and mispairing hybridization is either nearly zero or the mispairing event is even energetically “favoured”. In those “permissive” buffer conditions therefore the mispairing event may either be statistically equal to a 100% complementary hybridization and/or may even be statistically more likely. These “permissive” buffer conditions may then be used for PCR or other nucleic acid amplification techniques in which the mispairing of the two nucleic acid sequences is preferred. Without being bound to this theory, the insight that there are buffer conditions in which nucleic acid mismatches are favoured also seems to explain why mismatched nucleic acids are more frequently amplified in some cells. This allows for the hypothesis that, at least in certain cancers and other diseases, where nucleic acid mismatches play a role, it might be explained by the presence of buffer conditions such cells in which the energetic advantage lies with incomplete hybridization rather than 100% complementary hybridization. In this sense, the disclosed method can also be adapted to measure the energetic conditions of a cell medium, and in the case of a “permissive” cell medium composition, to predict the likelihood of disease development. Thus, in one embodiment the disclosed methods may be used to predict the chances to develop a disease originating form nucleic acid mispairings, such as one selected from the group consisting of cancer, lynch syndrome (Hereditary Nonpolyposis Colorectal Cancer, HNPCC), xeroderma pigmentosum, mutations leading to genetic disorders, cystic fibrosis, sickle cell anaemia, neurodegenerative disorders such as Huntington's disease and certain forms of spinocerebellar ataxia, Fanconi anaemia, hereditary breast and ovarian cancer syndrome (such as caused by mutations in BRCA1 and BRCA2 genes), and ataxia-telangiectasia.

In one embodiment the buffer medium is water or any buffer allowing a hybridization of the nucleic acid molecules tested, i.e. which provide the necessary ionic strength and environment to facilitate the hybridization of nucleic acids to complementary strands.

In some embodiments the buffer medium may comprise:
a) a salt selected from sodium chloride (NaCl), sodium citrate, and sodium phosphate, as well as any combination thereof;
b) a detergent selected from the group consisting of sodium dodecyl sulfate (SDS), polyethylene glycol tert-octylphenyl ether (Triton X-100), nonylphenoxypoly(ethyleneoxy) ethanol (Nonidet P-40 (NP-40)), octylphenoxypolyethoxyethanol (Igepal CA-630), Polysorbate 20 (Tween 20), 3-[(3-cholamidopropyl)dimethylammonio]-1-propanesulfonate (CHAPS), N-lauroylsarcosine sodium salt (Sarkosyl) and sodium deoxycholate (Deoxycholate), as well as any combination thereof;
c) a blocking agent selected from Denhardt's solution (comprising Ficoll, polyvinylpyrrolidone, and bovine serum albumin), salmon sperm DNA and herring sperm DNA, as well as any combination thereof;
d) a buffer such as Tris-HCl-buffer, HEPES-NaOH, saline-sodium citrate buffer, saline-sodium phosphate-EDTA buffer, as well as any combination thereof;
e) a chelating agent such as EDTA; and has a pH from 7.0 to 8.0.

In one embodiment the buffer medium is selected from the group consisting of standard SSC (Saline-Sodium Citrate) buffer, Denhardt's solution, Church buffer, formamide hybridization buffer, DIG Easy Hyb, HEPES buffer, and lysis buffer, as well as combinations thereof.

SSC (Saline-Sodium Citrate) buffer is a widely used buffer solution in molecular biology, especially in nucleic acid hybridization techniques. It provides the ionic strength and pH conditions necessary for the hybridization of DNA or RNA to complementary strands. The standard formulation for SSC buffer is as follows:
3 M NaCl (Sodium Chloride)
0.3 M Sodium Citrate (Tri-sodium citrate dihydrate)
pH adjusted to 7.0 with HCl

Denhardt's solution is a blocking agent used in molecular biology, particularly in nucleic acid hybridization techniques, to prevent non-specific binding of probes to the membrane or other surfaces. It helps to reduce background noise and increase the specificity of the hybridization signal. The standard formulation for Denhardt's solution is as follows:
2% (w/v) Ficoll 400
2% (w/v) Polyvinylpyrrolidone (PVP)
2% (w/v) Bovine Serum Albumin (BSA)

These components are dissolved in distilled water to create a 100× stock solution, which can be diluted to the desired working concentration.

Church buffer, also known as Church and Gilbert buffer, is a hybridization buffer used in molecular biology to enhance the sensitivity and specificity of nucleic acid hybridization reactions. It was developed by George M. Church and Wally Gilbert. This buffer provides an optimal environment for the hybridization of DNA or RNA probes to target sequences on membranes, reducing non-specific binding and background noise. The composition of Church buffer can vary slightly depending on the specific application, but the standard formulation typically includes:
1% Bovine Serum Albumin (BSA)
1 mM EDTA (Ethylenediaminetetraacetic acid)
0.5 M Phosphate Buffer (pH 7.2)
7% SDS (Sodium Dodecyl Sulfate)

Formamide hybridization buffer is a commonly used solution in molecular biology for nucleic acid hybridization, particularly in procedures like in situ hybridization, Southern blotting, and Northern blotting. The inclusion of formamide in the buffer helps to lower the melting temperature (Tm) of nucleic acid duplexes, allowing hybridization to occur at lower temperatures, which can be crucial for preserving the integrity of nucleic acids and improving hybridization specificity.

A typical formamide hybridization buffer might include:
50% Formamide
5×SSC (Saline-Sodium Citrate) Buffer
0.1% SDS (Sodium Dodecyl Sulfate)
0.02% Polyvinylpyrrolidone (PVP)
0.02% Ficoll
0.02% Bovine Serum Albumin (BSA)
Denhardt's Solution (sometimes included)
100 μg/mL denatured, sheared salmon sperm DNA (or other blocking DNA)

DIG Easy Hyb is a hybridization buffer designed for use in molecular biology applications, particularly in hybridization techniques that employ DIG (digoxigenin)-labeled probes. It is optimized to enhance the specificity and sensitivity of hybridization reactions, facilitating the detection of nucleic acids in various assays such as Southern blotting, Northern blotting, and in situ hybridization. While the exact composition of DIG Easy Hyb is proprietary, it typically includes components that promote efficient hybridization and stability of the DIG-labeled probes, such as:
Blocking Agents: To reduce non-specific binding.
Salts: To maintain ionic strength and stabilize nucleic acid duplexes.
Detergents: To reduce background noise.
Buffering Agents: To maintain optimal pH.

HEPES (4-(2-hydroxyethyl)-1-piperazineethanesulfonic acid) buffer is a zwitterionic buffering agent commonly used in biological and biochemical research due to its excellent ability to maintain a stable pH over a wide range of temperatures. In the context of nucleic acid hybridization, HEPES buffer is sometimes used to provide optimal conditions for the hybridization process, ensuring that the pH remains stable during the reaction. A typical HEPES hybridization buffer may include:
HEPES: 10⁻⁵⁰ mM (depending on the specific application)
NaCl: To maintain ionic strength, typically around 0.1-1 M
EDTA: 1-5 mM, to chelate divalent cations and prevent degradation of nucleic acids
Detergents: Such as SDS (0.1-1%) or other non-ionic detergents, to reduce background noise
Denhardt's Solution: Often included to block non-specific binding sites
Formamide: 0-50%, to lower the melting temperature (Tm) of nucleic acid duplexes, facilitating hybridization at lower temperatures.
Blocking Agents: Such as sheared salmon sperm DNA or BSA, to prevent non-specific probe binding.

As lysis buffer any buffer may be used, in particular in cases in which the nucleic acid needs to be separated from other biologic material, such as in case of a cell or tissue-sample. In one embodiment the lysis buffer may comprise 50 mM Tris-HCl, 22 mM EDTA and 1.2% Triton X-100, and the pH is 7.0-7.5. In another embodiment the lysis buffer may comprise 150 mM NaCl, 10 mM Tris-HCl (pH 7.4) and 0.25% Triton X-100.

In one embodiment a simple Tris-HCL-Buffer may be used comprising 50 mM Tris-HCl (pH 7.4) and 100 mM HEPES-NaOH (pH 8.5).

In another embodiment of the disclosure the nucleic acid of the probe, the control nucleic acid and/or the sample nucleic acid is selected independently from each other from the group consisting of DNA (Deoxyribonucleic Acid), RNA (Ribonucleic Acid), PNA (Peptide Nucleic Acid), XNA (Xeno Nucleic Acid), LNA (Locked Nucleic Acid), morpholino oligomers, 2′O-Methyl-RNA, phosphorothioate DNA or RNA, guanosine-rich oligonucleotides, and D-amino acid-based nucleic acids, as well as combinations thereof.

In another embodiment of the disclosure the fluorescence-label is selected from the group consisting of fluorescent dyes such as DAPI, ethidium bromide, SYBR Green, Alexa Fluor dyes, and CyDyes, Quantum Dots and lanthanide-based labels, as well as combinations thereof.

In another embodiment of the disclosure the probe is excited by light in the wavelength corresponding to the absorption wavelength of the fluorescence-label.

In another embodiment of the disclosure the chaotropic agent is selected from guanidinium chloride (GuHCl), guanidinium thiocyanate (GuSCN), phenol, formamide, urea, potassium ions (K⁺), lithium ions (Li⁺), and magnesium ions (Mg²⁺), as well as any combination thereof;

In another embodiment of the disclosure the excitation light is provided by light-source selected from the group consisting of a mercury arc lamp, a xenon arc lamp, an LED, a LASER, a metal halide lamp, a tungsten-halogen lamp, and a deuterium lamp, as well as combinations thereof.

In another embodiment of the disclosure the mispairing is a pairing selected from the group consisting of A-A, A-C, C-A, A-G, G-A, T-T, T-C, C-T, T-G, G-T, C-C, G-G, U-U, A-U, U-A, as well as combinations thereof, wherein A is adenosine, C is cytosine, T is tyrosine, G is guanine, and U is uracil.

In another embodiment of the disclosure the sample nucleic acid molecule may be derived from a cell-lysate, a biological sample such as a tissue sample, or a body fluid selected from the group consisting of blood, urine, saliva, sweat, lymph fluid, cerebrospinal fluid (CSF), gastric juice, pleural fluid, peritoneal fluid, synovial fluid (joint fluid), and sputum (phlegm), as well as combinations thereof.

In another embodiment of the disclosure the sample nucleic acid molecule is a nucleic target for use in the detection of a nucleic acid mispairing in diagnostics, research, or a therapeutic application in a biological and/or medical field.

In one aspect the present disclosure relates to a detection device as described in EP20169908.9 which is incorporated by reference hereinunder. In short, the detection device is characterized by a light source for the excitation light, and a microprocessor that is arranged for automatically receiving data from the detector. The detection device may be further characterized by comprising a beam splitter with high pass optical filter, or a dichroic mirror between the sample and the detector which differentiates the excitation energy from an emission energy to be measured by the detector. The detection device may be further characterized in that the optical element is an optical fibre. The detection device may be further characterized by comprising at least one focusing element between the light source and the sample which delivers the excitation light onto the sample. The detection device may be further characterized by comprising at least one beam collimating element between the sample and the optical element which collects the emitted light (emission energy) to the optical element.

In particular, the present disclosure concerns a method for detecting a target nucleic acid in a sample, comprising the following steps:
Bringing the sample in contact with a probe, that is a nucleic acid which can hybridize with the target, wherein, optionally, the probe may be labelled with a fluorescent molecule;
b. Irradiating the probe with exciting energy, preferably excitation light,
c. Detecting the emission energy from the sample, preferably emission light,
d. Calculating ΔE from the energy-difference from the sample in comparison to a standard which is the probe-target-pair of same length and chemical composition, but 100% complementary to each other allowing a perfect hybridization.

In another aspect the present disclosure pertains to a sensor vessel as described in detail in PCT/EP2022/083446 which is included by reference herein. In short, the vessel is characterized in comprising of at least a reaction chamber, a ferrule, a light conducting element, such as optical fibre (glass or plastic) with a light conducting core element and a sheath encapsulating the light conducting core element, wherein the ferrule comprises a detection buffer and a probe.

The sensor vessel combines several technical solutions:
Safe collection and storage of biological sample (in at least one embodiment being pathogenic);
Facilitation of a lysis of the biological sample and facilitation of the binding of the nucleic acid probe with the target nucleic acid molecule within the liquid;
Facilitation a light conduction from the optical detection device to the complex of the a least two hybridized nucleic acid molecules (e.g. the probe and the target), thereby producing as little as possible “light pollution” in form of reflection, absorption and scattering.

In one embodiment, the sensor vessel is a “single use” or “disposable” device, which poses specific requirements regarding cost-effective production without jeopardizing the optical quality of each device.

In order to perform the disclosed method, a number of energy-levels need to be determined.

Generally, the energy difference ΔE corresponds to energy-difference of the only probe emission and the emission light of the probe-target duplex and corresponds to the hybridization energy of the at least two hybridized nucleic acid molecules; wherein a mispairing of the at least two hybridized nucleic acid molecules is characterized by a decrease or increase of energy-difference as compared to a 100% complementary control. By then adding a chaotropic substance the energy-levels may be further increased and decreased allowing for a quantitative analysis of how many base mispairings have occurred. The addition of the chaotropic substance also reduces any other artefacts which may be measured.

This finding is surprising because prior art literature suggests that mispairings should always decrease stability energy of the hybridized complex, |ΔE^{H}|>|ΔE^{HM}|. However, it seems that at least in certain buffer conditions, this rule does not apply, and the overall energy of the hybridization complex may be actually reduced.

The theoretical background is explained for example in the review-article (Petruska J, Sowers L C, Goodman M F. Comparison of nucleotide interactions in water, proteins, and vacuum: model for DNA polymerase fidelity. Proceedings of the National Academy of Sciences. 1986 March; 83(6):1559-62.)

In one embodiment the sensor vessel is configured to be used with the detection device such as described in detail in DE 10 2019 132 525.0.

In some embodiments, in particular in case of biological samples comprising intact cells, a buffer may be chosen which breaks the cells open (“lysis function”) for use in the further detection reaction in order to analyze the nucleic acids within the cell, respectively, the biological sample. The biological sample may be a blood sample, a saliva sample and/or a tissue sample, or any other human body fluid or tissue sample, and therefore the cells therein need to be broken open in order to analyze the nucleic acid within. In one embodiment between at least 50% and up to 100% of the cells in a sample are “broken open”, in a preferred embodiment between 60% and 99%, in a further preferred embodiment between 70 and 85%.

Another role of the buffer is to promote the detection reaction itself and not interfere with the detection (“detection function”), for example the buffer must promote hybridization of target molecules with the sensor probes (for example a hybridization and/or binding of the sensor probe with the target molecule) and allow the analysis to take place. For the detection function, it is important, that the buffer solution is “optically clear” at the wavelength, where the biomarker molecules are active, e.g. between 300 nm-1100 nm, preferably between 500 nm-700 nm.

Another role of the buffer-solution is to facilitate the binding of the biomarker with the sensor-probe.

The detection buffer of the disclosure comprises:
a) (optional) a salt selected from sodium chloride (NaCl), sodium citrate, and sodium phosphate, as well as any combination thereof;
b) a detergent selected from the group consisting of sodium dodecyl sulfate (SDS), polyethylene glycol tert-octylphenyl ether (Triton X-100), nonylphenoxypoly(ethyleneoxy) ethanol (Nonidet P-40 (NP-40)), octylphenoxypolyethoxyethanol (Igepal CA-630), Polysorbate 20 (Tween 20), 3-[(3-cholamidopropyl)dimethylammonio]-1-propanesulfonate (CHAPS), N-lauroylsarcosine sodium salt (Sarkosyl) and sodium deoxycholate (Deoxycholate), as well as any combination thereof;
c) (optional) a blocking agent selected from Denhardt's solution (comprising Ficoll, polyvinylpyrrolidone, and bovine serum albumin), salmon sperm DNA and herring sperm DNA, as well as any combination thereof;
d) a buffer such as Tris-HCl-buffer, HEPES-NaOH, saline-sodium citrate buffer, saline-sodium phosphate-EDTA buffer, as well as any combination thereof;
e) a chelating agent such as EDTA;
f) a chaotropic agent selected from guanidinium chloride (GuHCl), guanidinium thiocyanate (GuSCN), phenol, formamide, urea, potassium ions (K⁺), lithium ions (Li⁺), and magnesium ions (Mg²⁺), as well as any combination thereof;
g) and has a pH from 7.0 to 8.0.

The concentration of the probe (e.g. the molecular probe, MB) depends on the size of the probe, the lifetime and signal intensity of the fluorochrome and the properties of the exciting light (e.g. wavelength and signal intensity of the LASER and loss of energy in the optical pathway). In some embodiments the concentration of the sensor probe was chosen to be between 0.5 μM and 20 μM, preferably between 1 μM and 10 μM, preferably between 2 μM and 5 μM, in one embodiment about 1 μM in case of a LASER with 0.25 mW of nominal power, fibre diameter of 200 μm and NA (0.22).

As fluorochrome one may use any of the following dyes: Methoxycoumarin, DyLight®, Alexa Fluor®, Brilliant Violet 421™, HiLyte Fluor™, DyLight®, Alexa Fluor®, Aminocoumarin (AMCA), BD Horizon™, Pacific Blue™, EviTag™ quantum dots-Lake Placid Blue, AMCyan, BD Horizon™, Cy2®, Chromeo™, DyLight®, Alexa Fluor®, FAM, Fluorescein Iso-thiocyanate (FITC), EviTag™ quantum dots-Adirondack Green, Chromeo™, HiLyte Fluor™, Alexa Fluor® (405, 488, 514, 532, 546, 555, 568, 633, 647, 660, 680, 700, 750, 790), EviTag™, quantum dots-Catskill Green, Pacific Orange™, HEX, EviTag™ quantum dots-Hops Yellow, Cy3®, 5-TAMRA, Phycoerythrin (PE), Tetramethyl Rhodamine Isothiocyanate (TRITC), EviTag™ quantum dots-Birch Yellow, Cy3.5®, Rhodamine Red-X 570 590, PE-Dyomics®, EviTag™ quantum dots-Fort Orange, ROX, Red 613, Texas Red®, PE-Texas Red®, EviTag™ quantum dots-Maple-Red Orange, Allophycocyanin (APC), Quantum Red, Cy5®, PE-Cy5®, SureLight® P1, PE-Dyomics®, Peridinin Chlorophyll (PerCP), IRDye® 700DX, PE-Cy5.5®, APC-Cy5.5®, TruRed, APC-Cy7®, Cy7®, PE-Dyomics®, DyLight®, PE-Cy7®, IRDye® 800RS, DAPI, Hoechst 33258, Hoechst 33342, SYTOX Blue, YOYO-1, SYTOX Green, TOTO-1, TO-PRO-1, Mithramycin, SYTOX Orange, Chromomycin A3, CyTRAK Orange™, Ethidium Bromide, Propidium iodide (PI), DRAQ5™ and DRAQ7™, and/or combinations thereof.

In one embodiment system comprising the nucleic acids and the buffer medium of the present disclosure is adapted to a LASER which emits light at a wave length of at least one of the wave lengths selected from 325, 360, 405, 407, 488, 514, 532, 543, 568, 595, 633, 635 and/or 647 nm. The nominal power of the LASER may be between 1 mW and 3 W, preferably 5 mW to 1 W, in some embodiments about 10 mW, 25 mW, 50 mW, 100 mW, 200 mW, 300 mW, and up to 1 W, 2 W, 3 W. Higher nominal power may lead to heating of the probe, lower nominal power usually does not generate enough signal. In general, the number of photons (and related with this the emitted optical power) which reach the carrier element should be between 2×10¹⁵ and 5×10¹⁵ photons/s, for example about 3.2×10¹⁵ photons/s. Of course, this may be further adapted to the concentration of immobilized probes and optical activity of the used fluorochrome. In one embodiment a 532 nm LASER (diode LASER) with nominal power applied to the sample of about 0.25 mW is used.

In one embodiment the probe is a molecular beacon (i.e. nucleic acid such as for example an aptamer or RNA-probe, MB) and/or a target-binding molecule (i.e. an antigen-binding molecule, such as an antibody, fab-fragment, anticalin, or the like). In another embodiment the probe is a linear nucleic acid.

In one embodiment the method according to the disclosure preferably a peak wavelength of the detected light is defined that is specific to a hybrid of the target and the probe, and if the emitted light has the peak wavelength, it is concluded that the target is in the sample. This method is based on the finding that each target-probe hybrid has a unique wavelength of emitted light, and allows for identifying any known target, in a patient's sample.

In particular, the peak wavelength Nem may be calculated from h*c/λ_{cm}=h*c/λ_{ref}−E₀, wherein λ_{ref} is a reference emission of the fluorescent dye without hybridization, h is the Planck constant, c is the speed of light and E₀ is the hybridization energy of the target-probe hybrid, as known from Santalucia J Jr, Proc. Natl. Acad. Sci. USA 95 (1998) and calculated in terms of Nearest-Neighbor (NN) model, where change of the energy is 0.0243 eV. In some embodiments the change of the energy is between 0.01-0.05 eV, preferably between 0.02-0.03 eV, however, in some embodiment the energy may be up to 0.1 eV or even up to 0.2 eV.

In this respect it is important to note that the energy change of the peak wavelength upon hybridization and/or binding is measured (depicted as a shift between energy levels, generally called “ΔE”).

This measurement is not to be confused with prior art methods based for example on fluorescence excitation and/or FRET. The measurement of the present disclosure rather relies on the reduction of total free energy of the complex as compared to the single molecules, which is reduced after hybridization and/or binding.

In another embodiment the measurement allows even the differentiation between single nucleotide polymorphisms (SNPs), such as for example 1, 2, 3, 4, 5, 6, 7, 8, 9, 10, or more mispairings as long as the overall molecule still hybridizes. This allows the differentiation between different mutants or strains, e.g. different viral mutants, cancer types, and/or MRSA-strains.

In case of larger numbers of target macromolecules, such as in nano-molar-range or, preferably, micro-molar-range or, preferably, millimolar-range, as it may be the case of biomarkers found for example in blood samples, a quantitative measurement offers beneficial accuracy/precision in comparison with diagnostic methods used now (such as selective electrodes, titration methods etc.).

In one aspect the disclosure pertains to labelling a biomarker with the sensor-probe. Such a biomarker may be a nucleic acid contained in a patient's sample. According to the present disclosure in case of nucleic acids the probe is a molecular beacon. The detection buffer supports the lysis of the biological sample and the hybridization process. It is not necessary to extract the target nucleic acid (RNA or DNA) in advance since the method is very sensitive and is able to detect less than 20, preferably less than 10 copies per test. However, depending on the sample obtained, extraction of the target nucleic acid by generally known methods and/or commercially available extraction kits may be applied. In addition, when a double-stranded nucleic acid (DNA or RNA) has to be detected a strand separation can be made in advance or simultaneously with the hybridization. This strand separation may be conducted e.g. chemically could be performed simultaneously, or with UV-light or by heat treatment to be made in advance.

Detecting single nucleotide polymorphisms (SNPs) or nucleic acid mispairings under in vivo conditions opens up several significant applications in biomedical research and clinical practice.

Thus, the present disclosure encompasses the following uses and applications:

### Disease Diagnosis and Personalized Medicine

Genetic Disorders: Identifying SNPs associated with genetic diseases allows for early diagnosis and personalized treatment plans.

Cancer: Detecting SNPs linked to cancer susceptibility or drug resistance helps in designing targeted therapies.

Infectious Diseases: Identifying genetic variations in pathogens helps in tracking outbreaks and selecting appropriate treatments.

### Pharmacogenomics

Drug Response: SNPs influencing drug metabolism or efficacy can guide personalized drug selection and dosage adjustments.

Adverse Drug Reactions: Predicting susceptibility to adverse drug reactions based on genetic variations improves patient safety.

### Population Genetics and Evolutionary Studies

Studying SNP frequencies in populations provides insights into genetic diversity, migration patterns, and evolutionary relationships.

Identifying genetic adaptations to environmental pressures helps in understanding human evolution and adaptation.

### Forensic Analysis

Using SNPs for individual identification and forensic DNA profiling enhances accuracy and reliability in criminal investigations and paternity testing.

### Monitoring Treatment Efficacy

Monitoring changes in SNP profiles during treatment helps in assessing treatment response and predicting outcomes.

### Non-Invasive Prenatal Testing (NIPT)

Detecting fetal SNPs from maternal blood allows for non-invasive prenatal screening for genetic disorders.

### Environmental Monitoring and Agriculture

Assessing genetic diversity and health of wildlife populations or agricultural crops using SNP analysis contributes to conservation and breeding programs.

In summary, the ability to detect SNPs and nucleic acid mispairings in vivo has transformative implications across medicine, research, and beyond, enabling more precise diagnostics, treatments, and understanding of genetic diversity and disease mechanisms.

In another aspect of the disclosure the method can be applied to analyse the influence of other macromolecules such as peptides, proteins, carbohydrates or lipids on nucleic acid duplexes in a patient. The inventive method may be used both for predictive a well as diagnostic purposes.

In case of larger numbers of target macromolecules, such as in nano-molar-range or, preferably, micro-molar-range or, preferably, millimolar-range, as it may be the case of biomarkers found for example in blood samples, a quantitative measurement becomes especially feasible.

As used herein the term “optically clear” refers to a material which shows a transmissibility of the light at least in the detection wavelength of 500 to 700 nm of at least 95% or more, preferably of 98% or more, most preferably of 99% or more, such as more than 99.9%, more than 99.95%, more than 99.99%. Optically clear also implies that the material does not show any fluorescent effects neither in itself, nor on the probe alone or bound to the target molecule (i.e. the biomarker). Furthermore, “optically clear” materials preferably do not absorb and/or scatter the light.

As used herein the term “optically inactive” refers to a material which shows weak (a few %, i.e. 0.1%, 0.5%, 1%, less than 5%, less than 2.5%) to no transmissibility of the light at least in the in the detection wavelength of 500 to 700 nm. For example, a transmission of 0.01% or less, preferably of 0.001% or less, most preferably of 0.0001% and even better less than the detection limit. Furthermore, the term “optically inactive” as used herein refers to a material which shows no other optical effects such as luminescence or phosphorescence upon excitation with light. According to the present disclosure “optically inactive” material is used to avoid any “light contamination”, e.g. any light which comes from sources apart from the sensor-probe.

### C. Uses in Disease Diagnosis

As disclosed, the ability to detect SNPs and nucleic acid mispairings in vivo has transformative implications across medicine, research, and beyond, enabling more precise diagnostics, treatments, and understanding of genetic diversity and disease mechanisms.

In particular, the presently disclosed methods have been specifically shown to be efficacious in diseases including tuberculosis.

### 1. Tuberculosis

Mycobacterium tuberculosis (MTB) is a transmissible human pathogen that can cause either latent (asymptomatic) infection or active TB disease (TB). Nearly one third of the world's population is estimated to be infected with MTB (including both latent and active infections), and while progress is being made in terms of control of drug-susceptible TB (Lonnroth et al., 2010 Lancet 375:1814-29), 9.4 million cases of TB were identified and treated world-wide in 2008 (WHO Global tuberculosis control: short update to 2009 report; In: Organization WH, editor. Geneva, Switzerland, 2009). Despite this progress, drug resistance in MTB, and particularly multi-drug resistant TB (MDR-TB, defined as TB due to MTB that is resistant to the first line drugs isoniazid and rifampicin) has emerged as a threat to control of TB both in the US and abroad (Gandhi et al., 2010, Lancet 375(9728):1830-43). Global surveillance systems for drug-resistance in TB are inadequate, and therefore data are insufficient to inform whether the incidence of drug-resistant/MDR-TB TB is rising or falling. However, of the estimated 440,000 cases of MDR-TB occurring in 2008, only 7% were identified and reported to WHO and of these only a fifth were treated according to WHO standards (WHO. Global tuberculosis control: short update to 2009 report. In: Organization WH, editor. Geneva, Switzerland, 2009), suggesting that the problem is under-recognized and its true scope is unknown. The Global Project on Anti-Tuberculosis Drug Resistance, reporting on data collected from 83 countries, found the prevalence of drug resistance in new cases of active TB to be approximately 11% (Wright et al., 2009, Lancet 2009 373(9678):1861-73). Furthermore, mathematical modeling suggests that population control of drug-sensitive TB disease does not imply control of drug-resistant strains. Rather, removal of patients with drug-susceptible disease from a population may replenish the pool of individuals who are fully susceptible to MTB infection with minority circulating drug-resistant strains (Cohen et al., 2004, Nat Med 10(10):1117-21). While MDR-TB appears under control in some countries, in other settings rates of MDR-TB among all cases of TB are alarming. For example, in two provinces in China and in nine countries of the former Soviet Union, >7% of all new cases of active TB disease were MDR-TB (Wright et al., 2009, Lancet 373(9678):1861-73). Although drug sensitive active TB disease can be cured in 6 months, treatment of MDR-TB requires use of costly, toxic, and often ineffective second-line antimicrobials for >24 months (Shah et al., 2007, Emerge Infect Dis 13(3):380-7), is associated with high rates of morbidity and mortality, and has been described as both an international public health emergency (WHO. Multi-drug and extensively drug-resistant TB (M/XDR-TB): 2010 global report on surveillance and response, 2010) and a threat to the goal of TB elimination in the US (see US Publication No. US20140349320A1).

Drug resistance in MTB is due primarily to single nucleotide polymorphisms in genes encoding key mycobacterial enzymes (Blanchard, 1996, Annu Rev Biochem 65:215-39). The rpoB gene encodes the β-subunit of bacterial RNA polymerase, which is the target of rifampicin (Campbell et al., 2001, Cell 104:901-12; Jin and Gross, 1988, J Mol Biol 202:45-58). Mutations in this gene account for over 95% of clinical cases of rifampicin resistance (Telenti et al., 1993, Lancet 341:647-50) and are commonly associated with the presence of MDR-TB (Geffen, 2010. Cepheid Gene Xpert diagnostic technology for TB. HTB South; Shah et al., 2007, Emerg Infect Dis 13:380-7). The most common mutations are at codon 531, 526, 516, 511, and 533. These mutations are described in Table 1.

TABLE 1

| Codon | | |
|---|---|---|
| (M. tuberculosis) | Mutation | Effect on Resistance |
| 531 | S531L | Most common; high resistance |
| 526 | H526Y/D | Moderate to high resistance |
| 516 | D516V | Variable resistance |
| 511 | L511P | Rare, low-level resistance |
| 533 | G533D | Low resistance, sometimes silent |

Simple and rapid detection of mutations in these codons would aid in the fast identification and diagnosis of drug-resistant TB.

### Experiments

### Example 1

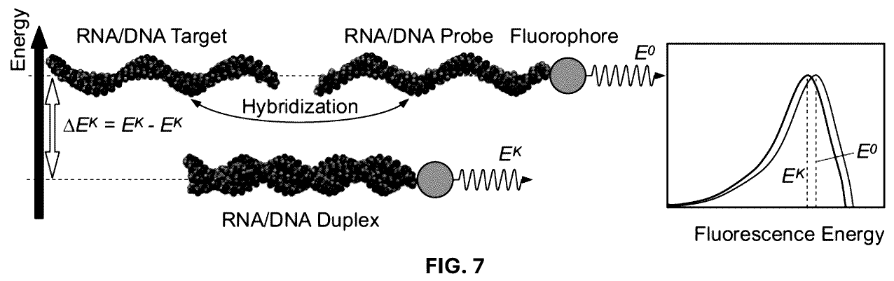

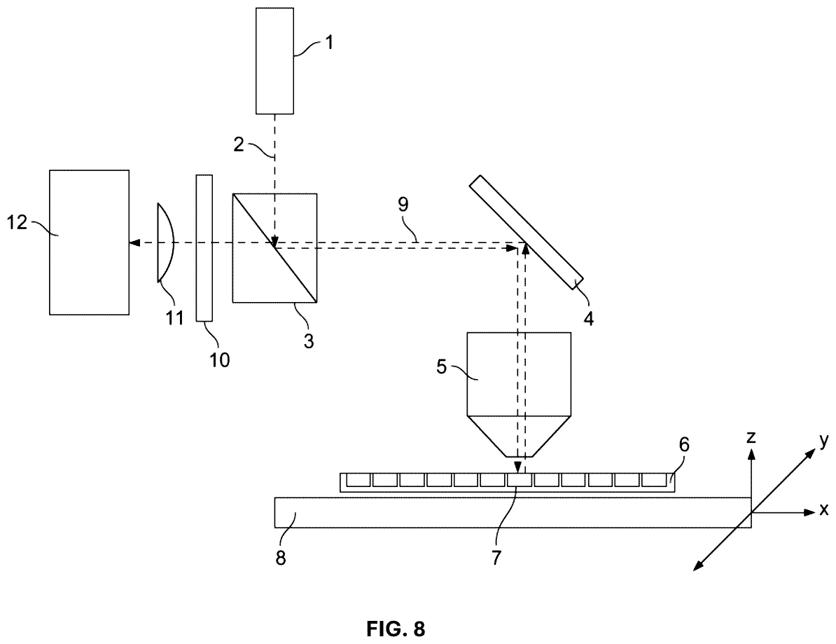

Schematically the experiment is shown on the FIG. 7, where the molecular probe labelled with the fluorophore molecule under optical excitation will emit light spectrum with peak position at the energy E⁰. Adding into reaction target sequence will change emission spectrum and shift the emission spectrum to the new position E^{K}. To measure the spectra E⁰ and E^{K}, the setup shown on the FIG. 8, can be used. Measured spectra to be processed, subtracted background and fitted with a function comprising double Gauss distributions. First gauss corresponds to emission of the dye molecule in monomer state (higher energies) and second in dimer state (low energies). For further analysis only the line (gaussian line) for the dye in monomer state will be used. The line position E^{K} will be analysed in function of time.

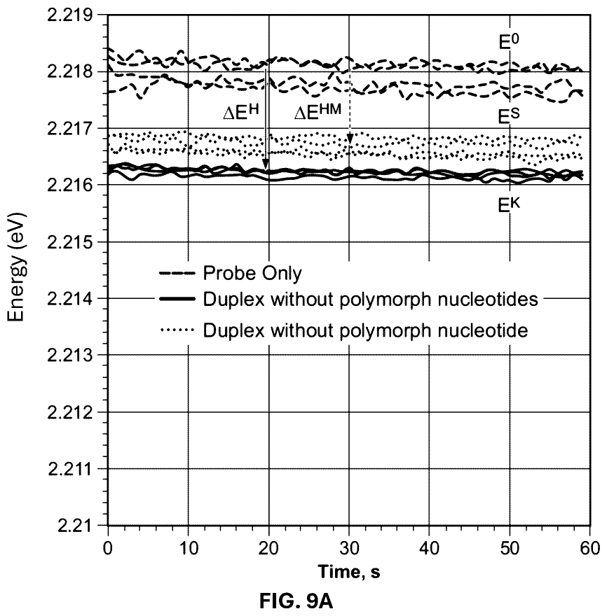

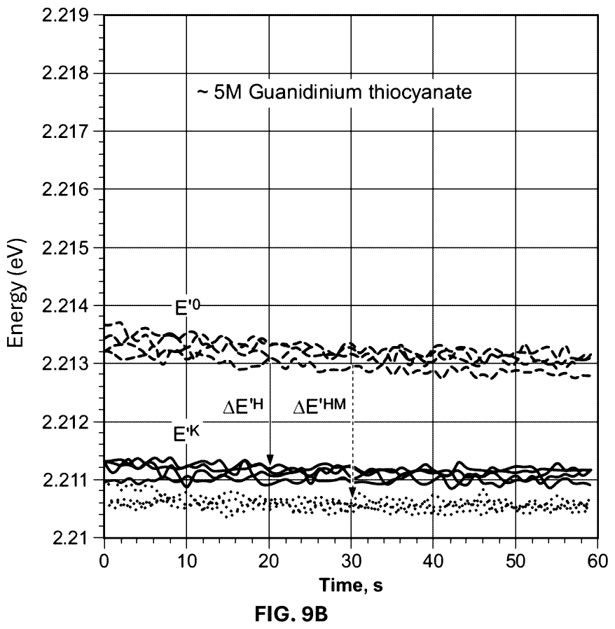

On the FIG. 9a) are shown the result of hybridization of the two types of the samples. First, 0.3 μM of 5′-AGA CCA GAA GAT CAG GAA CTC TA-3′ (SEQ ID NO: 1) molecular probe labeled with Rhodamine 6G dye hybridized with 0.3 μM perfectly matching target sequence 5′-TA GAG TTC CTG ATC TTC TGG TCT-3′ (SEQ ID NO: 2). And second type of the samples, where the same molecular probe hybridized with 0.3 μM of the target sequence comprising single polymorph nucleotide 5′-TA GAG TTC CTG ATT TTC TGG TCT-3′ (SEQ ID NO: 3). The hybridization reaction was carried in 50 mM Tris-HCl (pH 7.4) buffer. At first the only probe emission was measured dashed lines on the FIG. 9a). After adding target sequences (perfectly matching and comprising polymorph nucleotide) the ingredients were incubated for at least 30 minutes at room temperature. The emission (fluorescence) spectra of the incubated samples was measured for at least 60 sec with the rate of one spectrum per second. For each measured spectrum line position or emission energy was evaluated. Obtained dependencies are shown on the FIG. 9a) with solid E^{K} and doted E^{S} lines. As. it can be seen incubation of the molecular probe with perfectly matching target and with the target comprising single polymorph nucleotide results in the fluorescence energy change. The energy difference between the only molecular probe and the probe reach of ΔE^{H}˜1.8 meV or 21K for the perfectly matching target sequence and ΔE^{HM}˜1.6 meV or 18K for the target with polymorph nucleotide.

Obtained result shows that adding target sequences, which must increase free-energy of the sample, in spite to that, leads to decrease of the energy. The energy decrease is stronger for the perfectly matching target and slightly less for the target with polymorph nucleotide. The observation can be explained by hybridization of the molecular probe with the two targets, where duplex formed with perfectly matching target results in formation of more stable state comparably with the case where the duplex is formed with the target comprising single polymorph nucleotide. Other words, to the duplexes the perfect duplex must be heated to higher temperatures. The observation well agrees with theoretical models describing molecular hybridization.

The same experiment was carried in the lysis buffer containing ˜5 M guanidinium thiocyanate, 50 mM Tris-HCl, 22 mM EDTA and 1.2% Triton X-100, and the pH is 7.0-7.5, see FIG. 9b). Similarly as before, fluorescence of the only molecular probe in the lysis buffer was measured at first E⁰′. Afterwards the two types of the samples were created, first incubated with perfectly matching target sequence and second type where the molecular probe was incubated with the target comprising single polymorph nucleotide E′^{K} and E′^{S}. Similarly, as before, fluorescence energy changed its position, however the change for the imperfect duplex ΔE′^{HM}˜2.3 meV was stronger comparably with the perfect one ΔE′^{H}˜2.0 meV. The result is totally unexpected, because it shows that the less stable state become energetically favorable as the most stable duplex state. The possibility that the imperfect state may occupy lower energies as the perfect duplex state was already theoretically predicted. The free-energy difference between these states is small due to enthalpy-entropy compensation (Lumry®, Rajender S. Enthalpy-entropy compensation phenomena in water solutions of proteins and small molecules: a ubiquitous properly of water. Biopolymers: Original Research on Biomolecules. 1970 October; 9(10):1125-227.) observed in aqueous solutions. Exclusion of the water and substitution it with the 5M guanidinium thiocyanate as well as the broken hydrogen bond in the non-matching pair leads to increase of the entropy of the duplex which is not compensated. This leads to favoring the imperfect state over the DNA duplex formed with perfectly matching target.

### Example 2

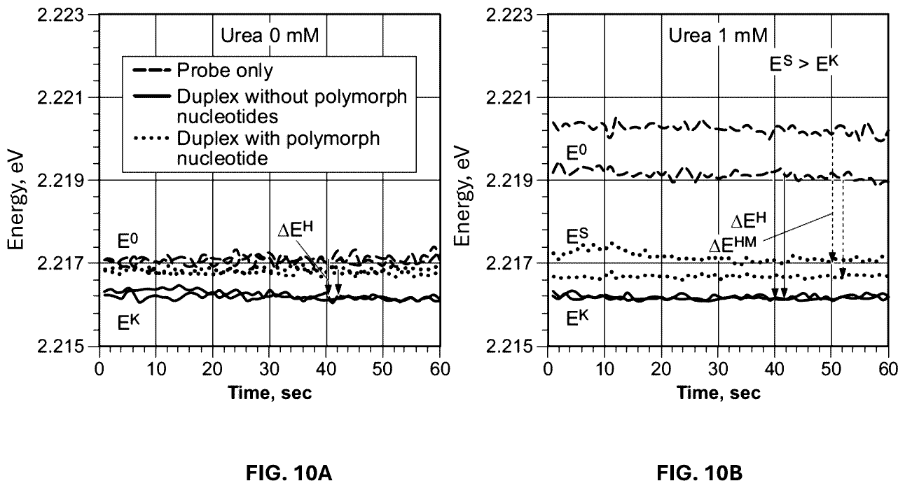

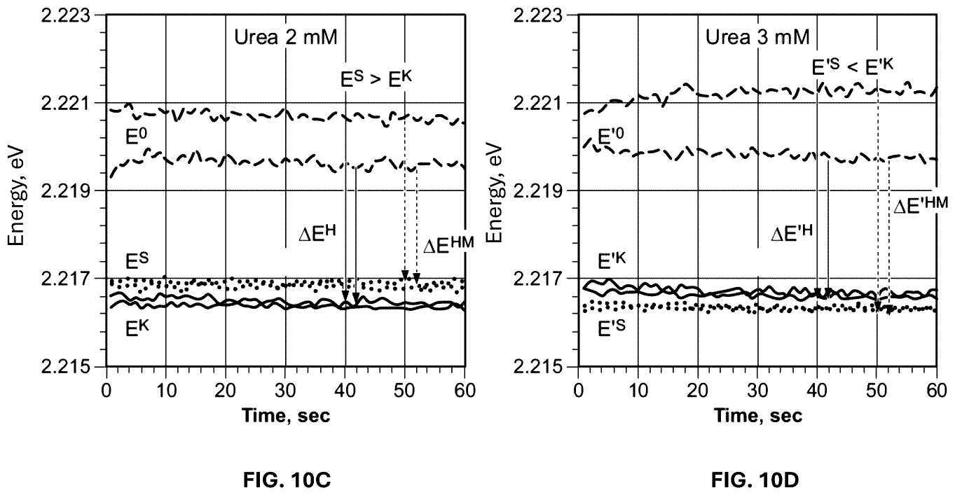

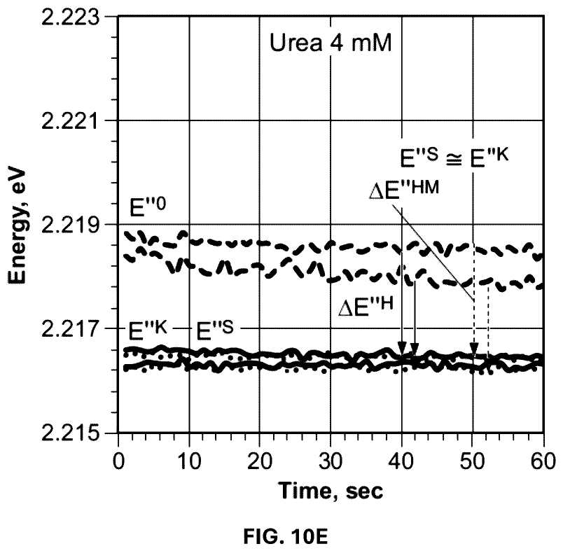

Another example is shown. on the FIG. 10. Here, hybridization of 0.3 μM of 5′-aga cca gaa gat cag gaa ctc ta-3′ (SEQ ID NO: 1) molecular probe and 0.3 μM perfectly matching target sequence 5′-TA GAG TTC CTG ATC TTC TGG TCT-3′ (SEQ ID NO: 2), as the first type of samples and the same molecular probe hybridized with 0.3 μM of the target sequence comprising single polymorph nucleotide 5′-ta gag ttc ctg att ttc tgg tct-3′ (SEQ ID NO: 3) as the second type of samples were performed in buffers comprising 0 mM, 1 mM, 2 mM, 3 mM and 4 mM of urea, FIG. 10 a)-e) respectively. Previously as before experiment was started by measuring the molecular probe fluorescence energy in the respective buffers, afterwards the strands were added into the respective set of the samples. For the 0 mM of urea, FIG. 10a), formation of the imperfect duplex was not observed also perfect duplex showed less energy change ˜1 meV comparably with the Tris buffer ΔE^{H}˜1.8 meV. Adding 1 mM and 2 mM of the urea, FIG. 10 b) and c) respectively shifted fluorescence energy of the samples comprising only the molecular probe into higher energies. Adding of the target sequenced (perfectly matching and containing single mismatching nucleotide) and >30 min incubation, resulted in significant lowering of the fluorescence energy E^{K} and E^{S} respectively. Here the perfect duplexes occupying lower energies comparably with the imperfect once, E^{S}>E^{K}. At around 3 mM of urea concentration, the energy of the molecular probe only continues to grow but for the hybridized samples the situation changes, see FIG. 10d). The imperfect duplexes observed at lower energies as the perfect once E′^{S}<E′^{K}, apparently this can be considered as the point where the water was completely substituted with the urea molecules. Father increase of the urea concentration to 4 mM, lead to decrease of the only probe energy comparable with the lower concentrations, as well as to the inability to resolve perfect and imperfect duplexes E″^{S}=E″^{K}.

### Example 3

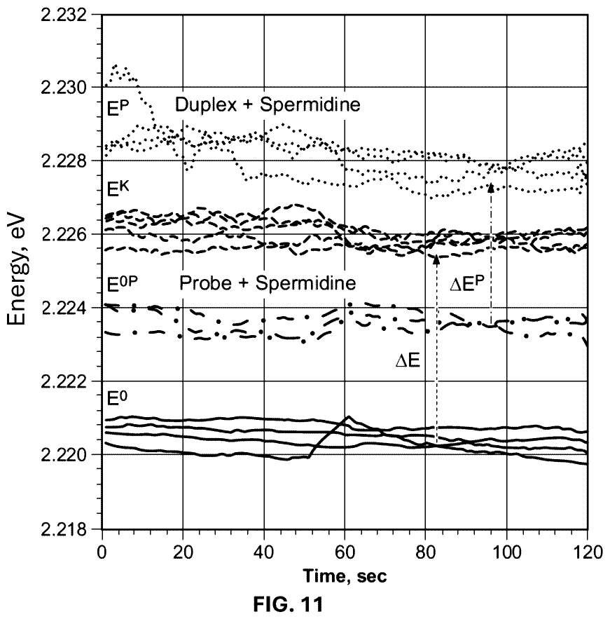

The last example is shown on the FIG. 11. Here, hybridization of the molecular probe (40 bp long, SEQ ID No. 6) 5′-GTG AGT TTG GGG AAA AAA AAT AAA ATA AAA ATG GCT TTC C-3′ labeled with Rhodamine 6G with perfectly matching target 5′-GGA AAG CCA TTT TTA TTT TAT TTT TTT TCC CCA AAC TCA C-3′ sequence (SEQ ID No. 7) and the mismatching (MM) target, central nucleotide (bold underlined), 50-CGCGAT AGACCAGAAAATCAGGAACTCTA ATCGCG-30 (SEQ ID NO: 8) in 50 mM Tris-HCl (pH 7.4) buffer was studied. As it can be seen the only molecular probe fluorescence occurring in higher energies comparably to the fluorescence of the 23 pb's long probe, FIG. 9a), E⁰˜2.221 eV (FIG. 11 solid line) vs. 2.218 eV respectively, this can be explained that adding longer nucleotides into the buffer causes significant perturbation for the buffer consequently the equilibrium between the buffer and the molecular probe will be reached at higher energies. Administering of the 0.3 μM of complementary target into the samples causes further grow of the fluorescence energy to the E^{K}˜2.226 eV, dashed line. The observed energy values can be used to estimate of each individual state especially such which are not possible to measure, for example the ground state of the buffer E^{B}. For the case of the only molecular probe in the Tris the energy equilibrium equation can be written as:

$${E}^{B} + {E}^{0} = 2.2 \; 21 eV , \qquad ( 1 )$$

Infusing of the same amount of the complementary target (the same length as the molecular probe), should double the E⁰, however it also triggers hybridization reaction, which decreasing the total energy on ΔE^{H}, so the energy equilibrium equation will look like:

$${E}^{B} + 2 {E}^{0} - Δ {E}^{H} = 2.226 eV , \qquad ( 2 )$$

where ΔE^{H} can be estimated theoretically and for the duplex it may reach ˜4.7 meV. Solving system of (1) and (2) equation estimated buffer energy is E^{B}˜2.211 eV and adding to the buffer 0.3 μM of the molecular probe increase the free energy on ˜10 meV.

Finally, the similar measurements were performed in presence of 5 mM spermidine. It is known that the spermidine and some other polyamines prefer to interact or bond to certain sites in DNA and also leading to conformational changes of the DNA. As it can be seen 5 mM of the spermidine changing fluorescence energy of the molecular probe E^{0P}˜2.2235 eV, dash-dotted line, as well as the position of duplex also is changed and shifted into higher energies to E^{P}˜2,228 eV. Repeating previous consideration, it is easy to conclude that the effective energy of the buffer comprising spermidine could be ˜2.2143 eV and the duplex melting temperature will be on ˜5K higher as for the duplex formed without spermidine. The conclusion agrees with the observation made from the Tm measurements. Although the method investigating energy states nucleic acids interacting with the molecules (polyamines and/or proteins) is disclosed for the first time

These methods are further described in the following citations, which are both incorporated herein by reference in their entirety (Rosencrantz, et al., “A method to measure molecular hybridization”, PLOS ONE 19(8): e0308084 (2024); Gottschalk, et al., “Formation of DNA duplexes in the presence of urea as a chaotropic agent”, bioRxiv, doi: 10.1101/2025.11.07.686900).

### Example 4

A similar experiment was conducted with tuberculosis. Oligonucleotides were purchased from biomers.net (Ulm, Germany). The MB used in the experiment was 5′-CGA CGA GGA GGA CCG CCA CGT GGT GGC ACA GGC CAA TTC GCC GAT CGA TGC GGA CGG TCG CTT CGT CG-3′ (SEQ ID NO. 9), labeled at the 5′-end with rhodamine 6G and at the 3′ end with the quencher BMN-Q620. The base pairs which are manipulated in the test strands are shown in bold and underline. A complementary target strand were prepared, which was a perfect match (PM) for the probe, 5′-CGC GGC TCG ACG AAG CGA CCG TCC GCA TCG ATC GGC GAA TTG GCC TGT GCC ACC ACG TGG CGG TCC TCC TCG TCG GCG-3′ (SEQ ID NO. 10).

A target strand containing a single mismatch (SM) was prepared (bold underlined), 5′-CGC GGC TCG ACG AAG CGA CCG TCC GCA TCG ATC GGC GAA TTG GCC TGT GCC ACC ACG TGG CGA TCC TCC TCG TCG GCG-3′ (SEQ ID NO. 11), and a target strand containing a double mismatch (DM) was prepared (bold underlined), 5′-CGC GGC TCG ACG AAG CGA CCG TCC GCA TCG ATC GGC GAA TTG GCC TGT GCC ACC ACG TGG CAA TCC TCC TCG TCG GCG-3′ (SEQ ID NO. 12). These represent mutations of codon 531 involved in resistant tuberculosis.

The MB was tested in LB1 buffer, comprising ˜5 M guanidinium thiocyanate, 50 mM Tris-HCl, 22 mM EDTA and 1.2% Triton X-100. Cell lysate was prepared by solubilizing pellet from 5×10⁶ cells of SLC-354 cell line (Ho\ddot{ }lzel Diagnostika Handels GmbH, Ko\ddot{ }ln) in 200 μL LB1 buffer. Each liquid sample initially comprised 5 μL of 1 μM solution of the MB. Later into the samples we added 1 μL of the PM, SM, or DM targets (5 μM in Millipore water) or 1 μL of Millipore water as a control.

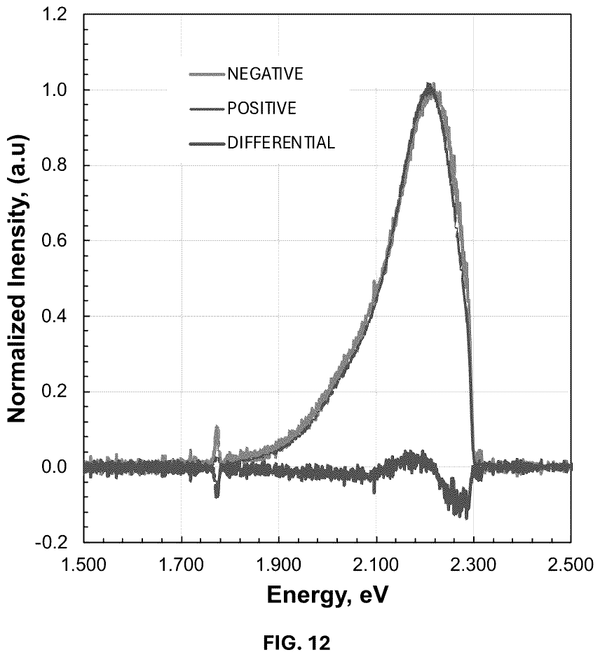

Each sample was measured in accordance with the following routine. Fluorescence was measured twice for the sample containing the MB with a time gap between these measurements of at least 20 min. Immediately after the second measurement, one of the targets or water was added into the solution and the fluorescence was measured again. The fourth and the fifths measurements were made at least 10 min and at least 30 min after target addition, respectively. All measurements were taken at room temperature and all solutions were protected against ambient light. This resulted in data reads for the molecular beacon (MB), perfect match (PM) with MB, single mismatch (SM) with MB, and double mismatch (DM) with MB. These values were normalized to 1. FIG. 12 provides a graph of the spectrum of just the MB (NEGATIVE) and the spectrum of the MB and PM (POSITIVE). These normalized values were subtracted from each other to yield the DIFFERENTIAL shown in FIG. 12. This differential confirms hybridization of the PM and MB.

The measurements were done using a custom-made micro-fluorescence device described herein and depicted in FIG. 8, in which the excitation light source was a 532-nm diode-pumped solid-state laser (5 mW emission power) passed through a 90:10 (Transmission: Reflection) beam splitter to give a final output of ˜0.5 mW. This was reflected into the optical fiber using the coupler. The other end of the optical fiber (cut and polished) was immersed into the analyte. Part of the fluorescence emitted by the MB in the analyte couples back into the excitation fiber, and 90% of the fluorescence intensity after the beam splitter and high-pass fluorescence filter is coupled into the optical fiber using the second coupler, so that fluorescence was measured by the spectrometer. The wavelength scale of the spectra was converted to eV for convenience. The setup works in near-field configuration, so any changes in the refractive index of a test liquid (or related effects) can be considered negligible. The laser excitation power delivered to the working volume was adjusted to exclude or at least limit laser-induced heating.

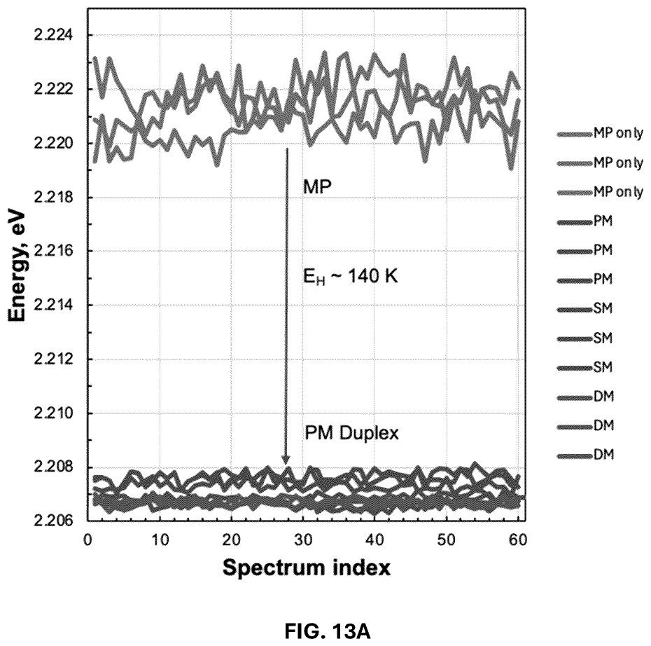

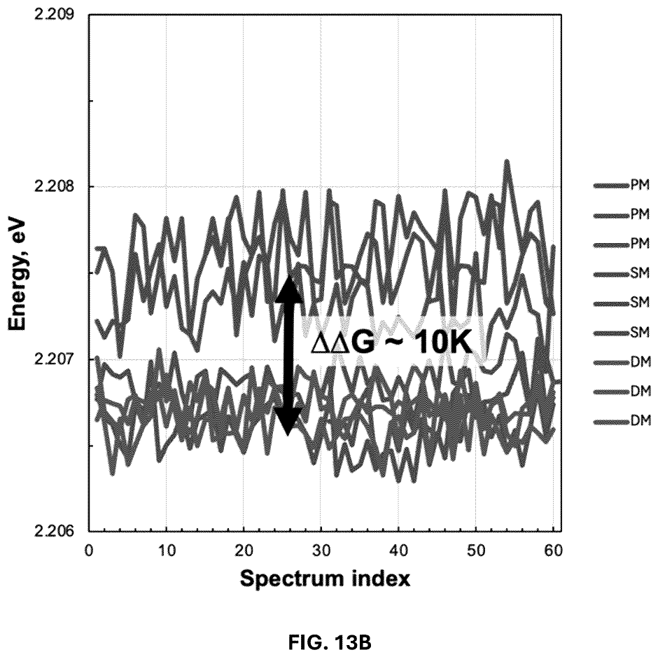

The line positions in Energy, eV were determined for the NEGATIVE and POSITIVE spectra as described above, shown in FIG. 13A. The difference in the line positions between the NEGATIVE and POSITIVE corresponds to the hybridization energy. In this case, the En was ˜140 K. Furthermore, the line positions of SM with MB and DM with MB were also plotted in FIG. 13B. A blown-up portion of this is shown in FIG. 13B. Here, the top lines represent the PM with MB readings, while the bottom lines represent the positive variants, namely SM with MB and DM with MB. The difference in the line values of the POSITIVE and POSITIVE VARIANTs was determined to be ˜10 K, giving a AAG value of the same. These graphs, FIGS. 13A and 13B, where they are broken up by MB only, PM (PM with MB), SM (SM with MB), and DM (DM with MB).

### Example 5

Herein is described an extended description of Examples 1 and 2. Oligonucleotides were delivered by “biomers.net: the biopolymer factory” (Ulm, Germany). The linear molecular probe (LMP) used in the experiments, comprises of a 23-nt probe 5′-AGA CCA GAA GAT CAG GAA CTC TA-3′ (SEQ ID NO. 13), labeled at the 5′-end with rhodamine 6G. Two target strands were prepared, one of which was a perfect match (PM) for the probe 5′-TAG AGT TCC TGA TCT TCT GGT CT-3′ (SEQ ID NO. 14) whereas the other contained a mismatching (MM), central nucleotide (bold underlined), 5′-TAG AGT TCC TGA TTT TCT GGT CT-3′ (SEQ ID NO. 15) and scramble strand (SS) as a negative control 5′-GCT ATG CGT ATT GTT CAC TTG TC-3′ (SEQ ID NO. 16). The LMPs were diluted to a final concentration of 0.1 μM in 50 mM Tris-HCl buffer (pH 7.4), and one of the three target strands was added such that the LMP concentration remained unchanged, while the target concentration was 0.2 μM. The 1:2 ratio of LMP to target was chosen to promote complete hybridization of the LMPs, and the fluorescence was expected to arise primarily from the duplex state. Urea was used as the chaotropic agent, with concentrations ranging from 0 mM to 4 mM in 1 mM increments. After preparation, samples were heated to 37° C. and then a1-lowed to cool to room temperature. All measurements were performed at room temperature, and all solutions were protected from ambient light throughout the experiment (for further information, see Gottschalk, et al., “Formation of DNA duplexes in the presence of urea as a chaotropic agent”, bioRxiv, doi: 10.1101/2025.11.07.686900, which is incorporated herein in its entirety).

Fluorescence measurements were performed using a custom-built micro-fluorescence setup described herein as depicted in FIG. 8. Linear molecular probes (LMPs) diluted in the sample were excited using a 532 nm diode-pumped solid-state laser with an emission power of 5 mW. The laser beam passed through a 90:10 (transmission: reflection) beam splitter, yielding an effective excitation output of approximately 0.5 mW. The reflected beam was coupled into an optical fiber via a fiber optic coupler. The other end of the fiber, which was cut and polished, was immersed directly into the analyte solution.

The excitation light stimulated fluorescence emission from rhodamine 6G dye molecules covalently attached to the LMP. A portion of the emitted fluorescence was recaptured by the same optical fiber used for excitation. After passing through the beam splitter and a high-pass fluorescence filter, approximately 90% of the fluorescence signal was directed into a spectrometer via a second optical coupler. Typical measurements were performed for one minute, with each spectrum accumulated for one second displayed and stored on the control computer.

The setup operates in a near field configuration, allowing any changes in the refractive index of the sample, or between samples, to be considered negligibly small. The excitation power delivered to the samples was carefully adjusted to minimize potential heating effects caused by laser irradiation. For convenience in analysis, the fluorescence spectra were converted to energy units (eV), and a schematic representation of the measurement results is shown in FIG. 7. In this schema, the LMP with an attached dye in its single stranded state occupies an energy level denoted as E⁰, and the dye emits a corresponding fluorescence spectrum. Upon hybridization with target nucleotides, the LMP forms a duplex that occupies a lower energy level (E^{K}), where E⁰<E^{K}. As a result, the fluorescence spectrum emitted by the rhodamine 6G dye is red-shifted relative to the single stranded state. The energy difference ΔE⁰=E⁰−E^{K} corresponds to the hybridization energy related with the formation of the duplex.

The wavelength scale of the spectra was convnvenience (Lakowicz, Principles of fluorescence spectroscopy. Boston, MA: Springer US 2006 Sep. 15.). Fluorescence intensity and its variations during the experiment were monitored solely as the indicator of sample quality. Even within the samrepared from identical stock solutions, fluorescence intensity could vary by up to ˜50%. Therefore, the analysis focused primarily on the peak positions of the fluorescence spectra.

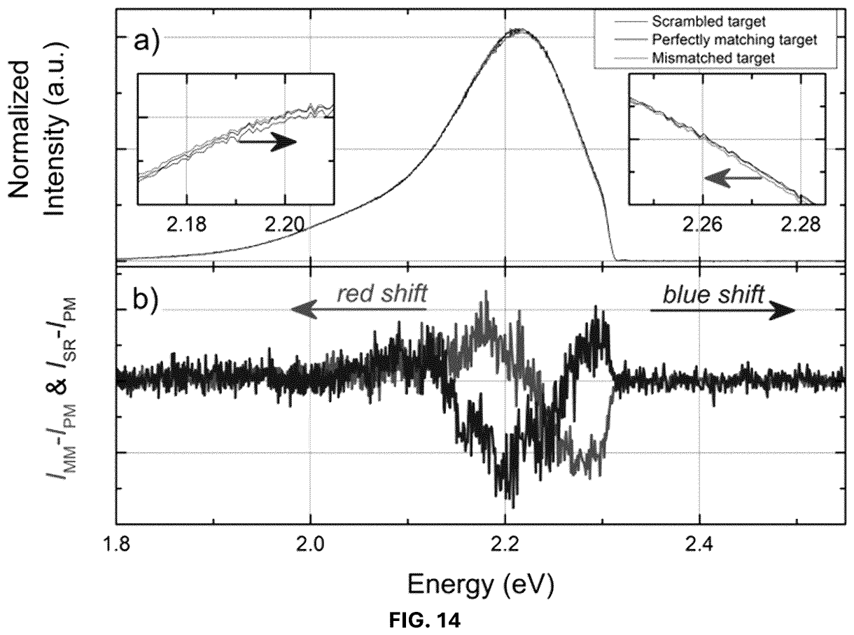

In FIG. 14, normalized fluorescence spectra are shown for three conditions: LMP with a scrambled target (single stranded, blue line, corresponding with blue shift), LMP hybridized with a perfectly matching target (duplex, black line, middle line), and LMP hybridized with a target containing a single nucleotide polymorphism (duplex with SNP; red line, corresponding with red shift). All measurements were performed in the presence of 2 mM urea. To more clearly resolve the spectral shifts, differential spectra were computed by subtracting the normalized fluorescence spectrum of the perfectly matched duplex from the spectra of the other two conditions (FIG. 14, bottom). These differential spectra show the direction of spectral shifts, either toward higher energy (blue shift) or lower energy (red shift). As seen, the differential spectrum for the scrambled target is blue shifted, while the duplex containing a polymorphic nucleotide exhibits a red shift relative to the perfect duplex.

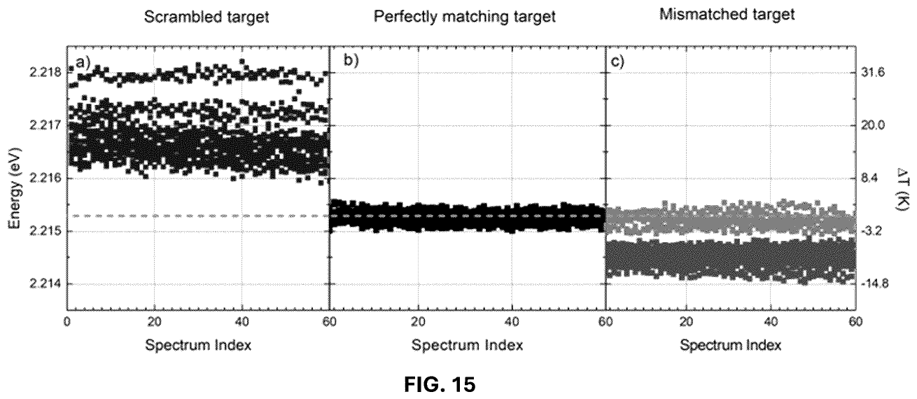

A detailed analysis of all experimentally obtained spectra was performed by fitting each with a sum of two Gaussian peak functions (Penzkofer, et al., “Fluorescence behaviour of highly concentrated rhodamine 6G solutions. Journal of luminescence. 37(2):61-72 (1987); Zehentbauer, et al., “Fluorescence spectroscopy of Rhodamine 6G: Concentration and solvent effects” Spectrochimica Acta Part A: Molecular and Biomolecular Spectroscopy, 121:147-151 (2014)). One Gaussian peak corresponded to the emission of rhodamine 6G in its monomeric state, while the other represented emission from dye dimers. Although only the monomer emission is of practical interest, accurately determining the spectral peak position requires a model that accounts for both components. Therefore, all spectra from all samples were fitted using this double peak model. The resulting monomer peak positions were grouped according to three conditions: LMP with a scrambled target (single stranded state; left box of FIG. 15), LMP hybridized with a perfectly matching target (duplex; middle box of FIG. 15), and LMP hybridized with a target containing a single nucleotide polymorphism (SNP; right box of FIG. 15).

The advantage of applying the fitting approach, particularly for broad and intense fluorescent emissions such as those exhibited by rhodamine 6G, is that it enables determination of the peak position with an accuracy higher the nominal resolution of the spectrometer. As shown in FIG. 14, the rhodamine 6G monomer emission line, centered near 2.21 eV, exhibits a spectral width of approximately 0.1 eV. Therefore, for rhodamine 6G spectra measured with common spectrometers, peak fitting allows the line position to be determined with an accuracy around 0.1 meV.

The energy reported by the LMP in the case where it reacts with the scrambled target varies over more than 2 meV in total, or over 20 K in thermal equivalent (see FIG. 15). This large energy spread is related to internal nonuniformity within the samples, caused by the presence of two nucleic strands that are unable to hybridize and form a duplex structure, and therefore cannot reduce their total energy. As shown below, no remarkable influence of urea on the energy is observed in this case, which is not surprising for the strongly perturbed and unstructured solutions.

For the double stranded duplex formed with the perfectly matching target, the energy reported by the LMP falls within a band approximately 0.5 meV wide, corresponding to about 6 K in thermal energy (see FIG. 15). Compared to the condition with the scrambled target, the band for the perfect duplex is narrower, but still relatively broad. This spread can be attributed to fluctuations in the concentrations of the LMP and the perfectly matching target between different samples. Similar to the previous condition, the presence of urea, despite its chaotropic nature, does not affect the energy of the LMP in the duplex state (see below). This is not surprising, as the duplex molecule represents the most stable and neutral state of DNA, with all hydrogen bonds fully engaged in stable pairing with the complementary strand. As a result, there are no unpaired bonds whose disruption could increase the duplex entropy in the presence of a chaotropic agent (SantaLucia, “A unified view of polymer, dumbbell, and oligonucleotide DNA nearest-neighbor thermodynamics” PNAS, 95(4):1460-1465 (1998)).

The final condition, where the LMP forms a double stranded structure with a single polymorphic nucleotide, demonstrates the most intriguing result. As shown in the rightmost box of FIG. 15, the emission energy of the LMP splits into two distinct bands, one corresponding to samples without urea, and the other to samples with urea. The energy levels for the mismatched samples without urea (top points in the right box of FIG. 15) overlap with those of the perfectly matched duplex and shows the similar band broadening. As can be seen from the figure, the values of the energy levels obtained for the duplexes with and without the SNP are very close. According to theoretical predictions Zuker, “Mfold web server for nucleic acid folding and hybridization prediction.” Nucleic acids research, 31(13):3406-3415 (2003)), the energy of the mismatched duplex should be about 0.2 meV (2 K) higher than that of the perfectly matched duplex. Considering that the accuracy of peak position determination is around 0.1 meV, this is not sufficient to resolve the energy levels. Moreover, broadening of the energy levels, with a width of ˜0.5 meV (6 K), makes it even more difficult to resolve these levels.

The duplex containing a polymorphic nucleotide, in the presence of the chaotropic agent urea, exhibits a lower energy compared to both the same duplex without the chaotropic agent and the perfectly matched duplex (Petruska, et al., “Comparison of nucleotide interactions in water, proteins, and vacuum: model for DNA polymerase fidelity” PNAS, 83(6):1559-1562 (1986)); Petruska, et al., “Comparison between DNA melting thermodynamics and DNA polymerase fidelity.” PNAS 85(17):6252-6256 (1988); Petruska, et al., “Enthalpy-Entropy Compensation in DNA Melting Thermodynamics” Journal of Biological Chemistry, 270(2):746-750 (1995); Goodman, “Hydrogen bonding revisited: geometric selection as a principal determinant of DNA replication fidelity” PNAS, 94(20):1049305 (1997)). The energy level broadening is approximately 0.7 meV (˜8 K), and the center of the band is shifted by about 0.9 meV (˜11 K) relative to that of the perfectly matched duplex. Similarly, as above, the energy level broadening is attributed to variations in the concentrations of the LMP and target strands, particularly when at least half of the target molecules remain unpaired (i.e., unhybridized). However, the distinct and significant red shift observed for the less favorable duplex, relatively to the perfectly matched duplex, cannot be explained by classical nearest-neighbor theory, which predicts a blue shift of about 0.2 meV (2 K).

Previous experiments described herein and reported in Rosencrantz, et al., “A method to measure molecular hybridization”, Plos one. 18(8): e0308084 (2024), which is incorporated herein in its entirety, reported a similar effect using molecular beacons, guanidinium thiocyanate at higher concentrations, and distinct experimental conditions This Example employed low concentrations of chaotropic agent urea (max. 4 mM) and LMP, precluding significant duplex destabilization. Consequently, urea showed no observable impact on the perfect matched duplex's energy state, which exists in its most stable configuration, rendering it largely impervious to environmental effects.

The presence of a single mismatched nucleotide introduces an unpaired hydrogen bond, enabling interaction with the surrounding environment. This site allows the duplex to interact with water molecules, inducing their local ordering and a consequent reduction in entropy, thereby increase the free energy of the duplex. However, the introduction of a chaotropic agent, such as urea, disrupts the ordered water structure, leading to an increase in system entropy and a reduction in free energy. This so-called “hydrophobic contribution to the dissociation energy” (Tanford, “Protein denaturation.” Advances in protein chemistry 23:121-282 (1968)) can yield to unexpected outcome. Under certain conditions, presence of the urea, thermodynamically less stable (mismatched) duplexes can become more energetically favorable than their perfectly matched duplexes, potentially resulting in mismatched duplexes preferential formation. Higher temperatures, however, reduce the dominance of this entropy driven hydrophobic effect, causing the system to revert to the expected behavior where more stable states (perfectly matched duplexes) exhibit higher melting temperatures than less stable mismatched duplexes.

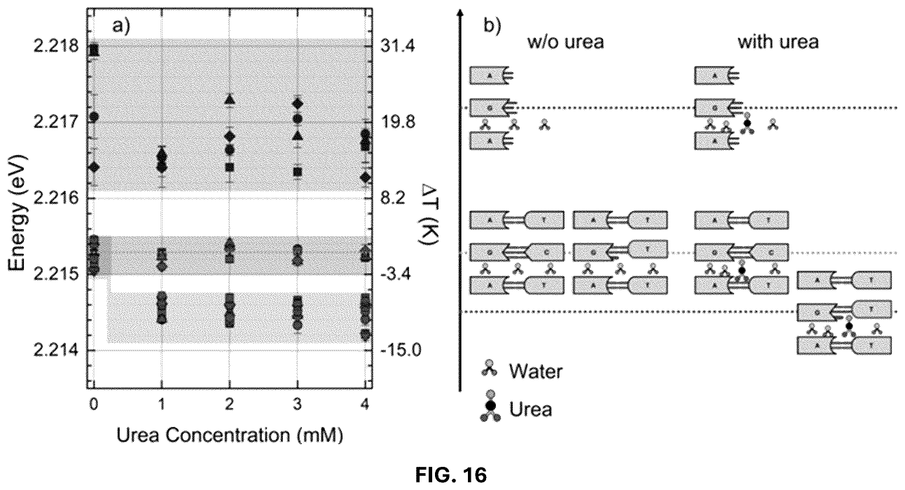

FIG. 16 shows the experimentally measured dependence of the emission energy of the LMP on the concentration of urea under three conditions: single stranded state with the scrambled target (top points, top zone), double stranded state with the perfectly matching target (middle points, middle zone), and double stranded state with the mismatched target (lower points, lower zone). As evident from the figure, the presence of urea has no remarkable influence on the energy of the LMP in either the single stranded state or the perfectly matched duplex. In contrast, the effect of urea on the mismatched duplex exhibits a binary character, the presence of urea activates the hydrophobic contribution, and after no further change in free energy is observed with increase of urea concentration. At lower concentrations of urea, one might expect a mixed state, where a portion of the duplexes containing SNPs are affected by the urea hydrophobic contribution, while the remaining portion are not. This may lead to a situation where the fluorescence will get its peak position at intermediate energies. But we do not believe that the mismatched duplex may exhibit a partial effect from the hydrophobic contribution. The right half of FIG. 16 summarizes these observations from the point of view of the nucleotides. The mismatched duplex, due to its sensitivity to environmental conditions via the unpaired hydrogen bond, responds to the presence of urea, a chaotropic agent that disrupts water structure, by occupying lower energy levels.

1. A method for analyzing a target nucleic acid in a biological sample comprising:
a) contacting the biological sample with a nucleic acid probe, wherein the nucleic acid probe is labelled with a fluorescent dye and adapted to hybridize with the target nucleic acid;
b) hybridizing the nucleic acid probe and the target nucleic acid to produce a hybridized complex;
c) irradiating the hybridized complex with an excitation light to excite the fluorescent dye;
d) observing, with a detector, an emission of light from the fluorescent dye, wherein the emission of light has an energy of peak fluorescence (E^{S});
e) determining if the hybridized complex has one or more mispaired base pairs, comprising:
i) calculating the hybridization energy difference of the nucleic acid probe hybridized to a control nucleic acid having 100% complementarity to the nucleic acid probe (ΔE^{H}) and the hybridization energy difference of the nucleic acid probe hybridized to the target nucleic acid (ΔE^{HM}), wherein ΔE^{H}=E⁰−E^{K} and ΔE^{HM}=E⁰−E^{S},
wherein E⁰ is a known energy of peak fluorescence of the nucleic acid probe;
wherein E^{K} is a known energy of peak fluorescence of the nucleic acid probe hybridized to a control nucleic acid having 100% complementarity to the nucleic acid probe;
ii) comparing |ΔE^{H}| and |ΔE^{HM}|, wherein if |ΔE^{H}|>|ΔE^{HM}|, the hybridized complex comprises at least one mispaired base pair.

2. The method according to claim 1, wherein the method further comprises calculating a change in the duplex melting temperature (ΔΔE) wherein ΔΔE=|ΔE^{H}|−|ΔE^{HM}|.

3. The method according to claim 1, wherein the method further comprises repeating steps a)-e) in the presence of a chaotropic agent; thereby determining chaotropic ΔE′^{H} and chaotropic ΔE′^{HM}, wherein ΔE′^{H}=E⁰−E^{K} and ΔE′^{HM}=E⁰−E^{S}.

4. The method according to claim 3, wherein the method further comprises calculating chaotropic ΔΔE′ by ΔΔE′=|ΔE′^{H}|−|ΔE′^{HM}| and calculating the number of mispaired base-pairs (n) by using the formula ΔΔE′˜n(Tₘ−Tₐ)ΔS,
wherein Tₘ is the temperature at which half of the hybridized complex dissociates;
wherein Tₐ is the temperature at which the nucleic acid probe is hybridized to the target nucleic acid; and
wherein ΔS is the entropy of a single broken hydrogen bond.

5. The method according to claim 1, wherein hybridizing the nucleic acid probe with the at least one target nucleic acid takes place in a buffer medium, wherein the buffer medium is water or any buffer allowing a hybridization of the nucleic acid molecules.

6. The method according to claim 5, wherein the buffer medium comprises:
a) a salt selected from sodium chloride (NaCl), sodium citrate, and sodium phosphate, or any combination thereof;
b) a detergent selected from the group consisting of sodium dodecyl sulfate (SDS), polyethylene glycol tert-octylphenyl ether (Triton X-100), nonylphenoxypoly(ethyleneoxy) ethanol (Nonidet P-40 (NP-40)), octylphenoxypolyethoxyethanol (Igepal CA-630), Polysorbate 20 (Tween 20), 3-[(3-cholamidopropyl)dimethylammonio]-1-propanesulfonate (CHAPS), N-lauroylsarcosine sodium salt (Sarkosyl) and sodium deoxycholate (Deoxycholate), or any combination thereof;
c) a blocking agent selected from Denhardt's solution (comprising Ficoll, polyvinylpyrrolidone, and bovine serum albumin), salmon sperm DNA and herring sperm DNA, or any combination thereof;
d) a buffer such as Tris-HCl-buffer, HEPES-NaOH, saline-sodium citrate buffer, saline-sodium phosphate-EDTA buffer, or any combination thereof; and
e) a chelating agent such as EDTA; and has a pH from 7.0 to 8.0, or any combinations thereof.

7. The method according to claim 5, wherein the buffer medium is selected from the group consisting of standard SSC (Saline-Sodium Citrate) buffer, Denhardt's solution, Church buffer, formamide hybridization buffer, DIG Easy Hyb, HEPES buffer, and a lysis-buffer, as well as combinations thereof.

8. The method according to claim 1, wherein the target nucleic acid is selected from the group consisting of DNA, RNA, PNA, XNA, LNA, morpholino oligomers, 2′O-Methyl-RNA, phosphorothioate DNA or RNA, guanosine-rich oligonucleotides, and D-amino acid-based nucleic acids, as well as combinations thereof.

9. The method according to claim 1, wherein the fluorescent dye is selected from the group consisting of fluorescent dyes such as DAPI, ethidium bromide, SYBR Green, Alexa Fluor dyes, and CyDyes, Quantum Dots and lanthanide-based labels, as well as combinations thereof.

10. The method according to claim 3, wherein the chaotropic agent selected from guanidinium chloride (GuHCl), guanidinium thiocyanate (GuSCN), phenol, formamide, urea, potassium ions (K⁺), lithium ions (Li⁺), and magnesium ions (Mg²⁺), as well as any combination thereof.

11. The method according to claim 1, wherein the nucleic acid probe is excited by light in the wavelength corresponding to the absorption wavelength of the fluorescent dye.

12. The method according to claim 11, wherein the light is provided by a light-source selected from the group consisting of a mercury arc lamp, a xenon arc lamp, an LED, a LASER, a metal halide lamp, a tungsten-halogen lamp, and a deuterium lamp, as well as combinations thereof.

13. The method according to claim 1, wherein the mispaired base pair is selected from the group consisting of A-A, A-C, C-A, A-G, G-A, T-T, T-C, C-T, T-G, G-T, C-C, G-G, U-U, A-U, U-A, as well as combinations thereof,
wherein A is adenosine, C is cytosine, T is tyrosine, G is guanine, and U is uracil.

14. The method according to claim 1, wherein the biological sample is a cell-lysate, a biological sample such as a tissue sample, or a body fluid selected from the group consisting of blood, urine, saliva, sweat, lymph fluid, cerebrospinal fluid (CSF), gastric juice, pleural fluid, peritoneal fluid, synovial fluid (joint fluid), and sputum (phlegm), as well as combinations thereof.

15. The method according to claim 1, for use in diagnostics outside the human or animal body or in medicine.

16. The method according to claim 1, wherein the target nucleic acid is a nucleic target for use in the detection of a nucleic acid mispairing in diagnostics, research, or a therapeutic application in a biological and/or medical field.

17. A method for analyzing a target nucleic acid in a biological sample in the presence of a sample molecule selected from a small molecule, peptide or protein comprising:
a) contacting the biological sample comprising the sample molecule with a nucleic acid probe, wherein the nucleic acid probe is labelled with a fluorescent dye and adapted to hybridize with the target nucleic acid;
b) hybridizing the nucleic acid probe and the target nucleic acid to produce a hybridized complex;
c) irradiating the hybridized complex with an excitation light to excite the fluorescent dye;
d) observing, with a detector, an emission of light from the fluorescent dye, wherein the emission of light has an energy of peak fluorescence (E^{P});
e) determining if the hybridized complex comprises an interference from the presence of the sample molecule, comprising:
i) calculating ΔE and ΔE^{P}, wherein ΔE=E^{K}−E⁰ and ΔE^{P}=E^{P}−E^{0P}, wherein E^{0P} is a known energy of peak fluorescence of the nucleic acid probe in the presence of the sample molecule;
wherein E⁰ is a known energy of peak fluorescence of the nucleic acid probe without the sample molecule;
wherein E^{K} is an energy of peak fluorescence of the nucleic acid probe hybridized to a control nucleic acid having 100% complementarity to the nucleic acid probe without the sample molecule;
ii) comparing |ΔE| and |ΔE^{P}|, wherein if |ΔE|≠|ΔE^{P}|, the hybridized complex comprises an interference from the sample molecule.

18. The method according to claim 17, wherein the interference is selected from the group consisting of bending of the nucleic acid, looping of the nucleic acid, altering accessibility and compaction of the nucleic acid, unwinding of the nucleic acid, supercoiling the nucleic acid, and modify the nucleic acid, as well as combinations thereof.

19. A method for detecting a drug-resistant tuberculosis infection in a subject, comprising steps of:
a) obtaining a biological sample from a subject;
b) contacting the biological sample with a nucleic acid probe, wherein the nucleic acid probe is labelled with a fluorescent dye and adapted to hybridize with at least one target nucleic acid;
c) hybridizing the nucleic acid probe and the target nucleic acid to produce a hybridized complex;
d) irradiating the hybridized complex with an excitation light to excite the fluorescent dye;
e) observing, with a detector, an emission of light from the fluorescent dye, wherein the emission of light has an energy of peak fluorescence (E^{S});
f) determining if the hybridized complex has one or more mispaired base pairs, comprising:
i) calculating ΔE^{H} and ΔE^{HM}, wherein ΔE^{H}=E⁰−E^{K} and ΔE^{HM}=E⁰−E^{S};
wherein E⁰ is a known energy of peak fluorescence of the nucleic acid probe;
wherein E^{K} is a known energy of peak fluorescence of the nucleic acid probe hybridized to a control nucleic acid having 100% complementarity to the nucleic acid probe;
ii) comparing |ΔE^{H}| and |ΔE^{HM}|, wherein if |ΔE^{H}|>|ΔE^{HM}|, the hybridized complex comprises at least one mispaired base pair;
wherein the presence of one or more mispaired base pairs in the hybridized complex is indicative of a drug-resistant tuberculosis infection in the subject.

20. The method of claim 19, wherein the at least one target nucleic acid comprises at least one of codons 531, 526, 516, 511, and 533 of the rpoB gene of Mycobacterium tuberculosis.


SEQUENCE LISTING (8 sequences)

SEQ ID NO: 1 - DNA, 23 bp, synthetic construct
  source 1..23: mol_type = other DNA
agaccagaagatcaggaactcta

SEQ ID NO: 2 - DNA, 23 bp, synthetic construct
  source 1..23: mol_type = other DNA
tagagttcctgatcttctggtct

SEQ ID NO: 3 - DNA, 23 bp, synthetic construct
  source 1..23: mol_type = other DNA
tagagttcctgattttctggtct

SEQ ID NO: 4 - DNA, 40 bp, synthetic construct
  source 1..40: mol_type = other DNA
gtgagtttggggaaaaaaaataaaataaaaatggctttcc

SEQ ID NO: 5 - DNA, 40 bp, synthetic construct
  source 1..40: mol_type = other DNA
ggaaagccatttttattttattttttttccccaaactcac

SEQ ID NO: 6 - DNA, 35 bp, synthetic construct
  source 1..35: mol_type = other DNA
cgcgattagagttcctgatcttctggtctatcgcg

SEQ ID NO: 7 - DNA, 35 bp, synthetic construct
  source 1..35: mol_type = other DNA
cgcgatagaccagaagatcaggaactctaatcgcg

SEQ ID NO: 8 - DNA, 35 bp, synthetic construct
  source 1..35: mol_type = other DNA
cgcgatagaccagaaaatcaggaactctaatcgcg